(19) United States
(12) Patent Application Publication
(10) Pub. No.: US 2008/0208179 A1
(43) Pub. Date: Aug. 28, 2008

(54) METHODS OF INCREASING SKIN PERMEABILITY BY TREATMENT WITH ELECTROMAGNETIC RADIATION

(75) Inventors: Kin F. Chan, San Jose, CA (US); Basil M. Hantash, East Palo Alto, CA (US); Vikramaditya P. Bedi, Redwood City, CA (US); George Frangineas, Fremont, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 11/925,693

(22) Filed: Oct. 26, 2007

Related U.S. Application Data
(60) Provisional application No. 60/863,113, filed on Oct. 26, 2006.

(51) Int. Cl. A61B 18/20; A61K 31/375

(52) U.S. Cl. 606/9; 514/474

(57) ABSTRACT

Methods of treating tissue with fractional laser radiation are disclosed. The fractional laser treatment methods reversibly increase skin permeability while maintaining a substantially intact stratum corneum and producing alterations within the epidermis and dermis. The alterations in the epidermis and dermis can include necrosis and/or coagulation. The alterations in the epidermis can include the creation of a plurality of pores in the stratum corneum and/or the creation of vacuoles in the layers of the epidermis below the stratum corneum. The fractional laser treatment methods disclosed herein can be used to provide treatments to the skin, to increase permeation of active substances into or through tissue, to deliver active substances locally or systemically, and to control the delivery of active substances.

### CROSS-REFERENCE TO RELATED APPLICATION(S)

This application claims priority under 35 U.S.C. 119(e) to U.S. Provisional Patent Application Ser. No. 60/863,113, “Methods of Increasing Skin Permeability by Treatment with Electromagnetic Radiation”, filed Oct. 26, 2006. The subject matter of the foregoing is incorporated herein by reference in its entirety.

### FIELD OF THE INVENTION

This invention relates generally to methods for increasing the permeability of tissue by irradiating it with fractional laser radiation. More particularly, it relates to fractional laser radiation treatment methods which increase the permeability of skin, and can be used to provide therapeutic and cosmetic treatments to the skin alone or in conjunction with active substances, to deliver active substances locally and systemically, and to control the delivery of active substances topically.

### BACKGROUND OF THE INVENTION

Poor permeation of many active substances into and through the skin often limits the utility of the topical route of administration and of topical formulations of active substances. Various methods exist in the art for increasing the permeability of the skin or for increasing the ability of an active substance to permeate the skin. Chemical enhancers can be used to reduce the barrier function of the skin or to alter the properties of the active substance so as to allow the active substance to better partition into the skin. These chemical modifiers can be quite irritating to the skin, and may not increase permeability adequately to allow therapeutic levels of many active substances to permeate the skin.

Energy-driven methods of increasing skin permeability have been developed, including electroporation and iontophoresis. Electroporation involves the use of relatively high electrical voltages over short periods of time to decrease the barrier function of the skin. Iontophoresis involves the use of relatively low electrical currents over a longer period of time to drive charged particles across the skin. Sonophoresis involves the use of ultrasound to drive active substances across the skin. The utility of these techniques is limited, as iontophoresis and electroporation are effective only with active substances that are stable in the presence of electrical currents, and all three methods increase skin permeability only during the period of time the treatment is applied.

Various methods of substantially avoiding or removing the barrier function of the skin have also been used. Microneedles, composed of arrays of very fine needles which pierce the upper layers of the skin to create holes through which active substances can penetrate, are considered minimally invasive. However, microneedles can be difficult to manufacture, and it can be difficult to position them within the skin so as to allow adequate permeation of active substances. Additionally, using microneedles can produce contaminated sharps, which pose a contamination threat and a medical waste disposal problem.

Various methods have also been used to ablate the stratum corneum, the outermost or uppermost layer of the skin, which poses the greatest barrier to permeation for many active substances. A large disadvantage of using ablative methods is increased risk of infection. Stratum corneum ablation techniques include suctioning, dermabrasion, radiofrequency thermal ablation, and laser ablation. Suctioning involves forming a small blister on the skin (usually with a vacuum), and removing the upper surface of the skin, thereby forming an area of skin without stratum corneum and allowing an active substance to readily permeate into and through the remaining skin layers. With suctioning, it is difficult to control the thickness of the blister created. Also, this technique produces relatively large areas of ablation that can take a long time to heal, resulting in an open portal for infection as well as active substances. As traditionally practiced, radiofrequency thermal ablation requires that an array of tiny, closely spaced electrodes be placed against the skin while an alternating current at radio frequency is applied to each microelectrode, thereby ablating the outermost layer of the skin. Control of the depth of ablation is difficult with this technique, and the need to place the microelectrodes directly in contact with the skin limits its utility.

Electromagnetic radiation, particularly as produced by lasers, has been applied directly to the skin for treatment of dermatological conditions, for skin resurfacing, to reduce or eliminate wrinkles, and to combat the effects of aging in the skin. Beyond treatment of the skin, electromagnetic radiation therapy has been used to increase the rate of wound healing, to reduce pain, to treat inflammatory conditions, as well as to reduce residual neurological deficits following stroke. When used for skin resurfacing, the effect of electromagnetic radiation on skin is primarily to heat the skin, producing coagulation, cell necrosis, melting, welding and ablation, among other effects. Treatment with electromagnetic radiation can generally be divided into ablative and nonablative treatments.

Ablation of the stratum corneum with electromagnetic radiation has been used for skin resurfacing and to perforate the skin to allow delivery of active substances and the removal or monitoring of biological fluids or gasses. U.S. Pat. No. 4,775,361 claims to describe a method of facilitating percutaneous transport by ablating the stratum corneum with pulsed laser radiation. The premise behind this invention is that the stratum corneum is the main barrier to permeation of active compounds, and the invention uses pulsed laser radiation to completely remove the barrier of the stratum corneum while avoiding penetration of the laser radiation into the viable epidermis. U.S. Pat. No. 4,775,361 does not discuss the use of nonablative laser radiation, nor does it discuss the use of fractional laser treatments.

The use of nonablative electromagnetic irradiation of the skin has been suggested to increase skin permeability by altering the lipid and protein molecules present in the stratum corneum, by producing heat, and by producing pressure waves.

U.S. Pat. No. 5,021,452 is directed to methods of applying low-power laser radiation of wavelengths between 600 and 1100 nanometers to tissue in combination with exogenously applied ascorbate to increase the cellular uptake of ascorbate and is useful in promoting wound healing.

U.S. Pat. No. 5,658,892 is directed to a method of increasing delivery of a compound from an exterior region to an interior region of a cell without causing destruction or cell death by using impulse transients. According to the patent, impulse transients can be generated using a pulsed laser, and can induce a time-dependent permeability of the exposed cell membrane.

With traditional electromagnetic radiation treatments, a large region of tissue is broadly irradiated by continuous or pulsed radiation, which heats the entire volume of tissue to bring about the desired effects. These broad, bulk treatments result in undesirable side effects such as pain, prolonged erythema, swelling, extended healing times, infection, and scarring. More recently, fractional electromagnetic radiation treatments have been used which involve the generation of a number of discrete treatment zones within a larger treated region of tissue. As with bulk treatments, the effects of the electromagnetic radiation on the tissue can include coagulation, cell necrosis, melting, welding, retraction, ablation, and alteration of the extra-cellular matrix, but only a limited portion of the tissue will experience these effects. The depth and degree to which these effects are created within the treatment zones is determined by controlling the treatment parameters used, such as local irradiance, local fluence, pulse energy, treatment zone size and treatment zone density. U.S. Pat. No. 6,251,100 describes methods for increasing skin permeability using a laser beam to perforate the stratum corneum to reduce or eliminate its barrier function, or to alter the stratum corneum to reduce or eliminate its barrier function and increase permeability without ablating, or by merely partially ablating the stratum corneum. Claimed methods of altering skin permeability according to this patent involve focusing a laser beam at the skin with sufficient energy fluence to alter the skin at least as deep as the stratum corneum but not as deep as the capillary layer. U.S. Pat. No. 6,419,642, describes methods for increasing skin permeability using a laser beam to perforate, ablate or alter one or more layers of the skin. This patent claims methods of introducing a substance into a living body comprising forming an area on the stratum corneum having enhanced permeability through to the capillary layer by irradiating the skin with subablative laser energy without substantially ablating the skin, and introducing the substance into the body by bringing it in contact with the area of enhanced permeability. To allow permeation of locally acting anesthetics, the patent describes perforating or altering the skin through the stratum corneum but not necessarily as deep as the capillary later. To allow permeation of other substances, the patent describes making perforations or alterations in the skin which do not penetrate as deep as the capillary layer, and which penetrate only the outer surfaces, such as the stratum corneum or both the stratum corneum and the epidermis.

United States Patent Application Publication Number US 2006/0004347 discusses methods of creating and differentiating types of “islets” in the skin, namely optical islets, thermal islets, damage islets, and photochemical islets. It states that the creation of thermal islets can be used to produce an increase in the permeability of the stratum corneum. Thermal islets are reported to define permeation pathways which can extend through or mostly through the stratum corneum and stratum lucidum layers, while the penetration of a cosmetic or therapeutic agent applied in this manner can be superficial and remain just below or within the stratum corneum, or can be deeper into the interior layers of the epidermis or dermis and, possibly, into the blood stream. This increase in the permeability of the stratum corneum is reported to last up to 2 hours. The patent claims a method of transdermal drug delivery of a topical preparation by applying optical energy to a portion of the stratum corneum to produce a multiplicity of thermal islets, where the thermal islets are heated to a temperature which causes an increase in the permeability of the stratum corneum, and a portion of the topical preparation diffuses across the portion of the stratum corneum during the application of the optical energy. Proposed advantages of the disclosed treatments include the ability to terminate the region of heating near the epidermal-dermal boundary instead of deeper in the dermis, the ability to produce permeability paths of less than 50 micrometers in depth to avoid damage to viable layers of the epidermis, and the ability to reduce or eliminate pain and discomfort of the patient by using less invasive treatments. The application also states that damage islets can be created to increase skin permeability by heating the tissue to temperatures higher than 100° C. to create small holes in the stratum corneum and so uses the electromagnetic radiation treatment to ablate, vaporize, or remove portions of the stratum corneum, increasing its permeability until those layers of the stratum corneum are replaced.

Thus, there remains a need for methods of increasing the permeability of the skin, and for increasing permeability of active substances into and through the skin using fractional laser treatments such as can be used for skin resurfacing which are capable of producing significant levels of alteration in the epidermis and dermis (i.e., necrosis, coagulation, pores and vacuoles), while also maintaining a portion of the barrier properties of the stratum corneum by maintaining a substantially intact stratum corneum.

### SUMMARY OF THE INVENTION

Fractional laser radiation treatments have been found which reversibly increase skin permeability while maintaining a substantially intact stratum corneum and producing alterations in the epidermis and dermis layers of the treated skin. The alterations produced in the epidermis and dermis can include necrosis and/or coagulation. The alterations produced in the epidermis can include the creation of a plurality of pores in the stratum corneum and/or the creation of vacuoles in the layers of the epidermis below the stratum corneum. The plurality of pores can be limited in depth to less than the full thickness of the stratum corneum (e.g., the pores do penetrate into the layers of the epidermis below the stratum corneum). The fractional laser treatments of the present invention which maintain a substantially intact stratum corneum can increase uptake of active substances while maintaining a substantial portion of the barrier function of the treated region of skin as compared to the barrier function of a normal, untreated region of skin. The fractional laser treatments described herein, which increase skin permeability while maintaining a substantially intact stratum corneum and producing alterations in the epidermis and dermis, can be used to treat the skin, to increase the permeation of active substances into and through the skin, to deliver active substances locally or systemically, and to control the delivery of active substances topically. The fractional laser treatments can be used to provide prophylactic, cosmetic, and/or therapeutic treatments of skin, alone or in combination with one or more than one active substance. An active substance in the form of a cosmetic and/or a pharmaceutical composition can be applied to the skin before, during and/or after the laser treatment, and can be applied once, repeatedly or continuously during treatment.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a drawing that illustrates the structure of the skin.

FIG. 2 is a drawing of a device capable of providing both positive pressure and vacuum while delivering laser radiation to the skin.

FIG. 3 is a series six of photographs of the surface of skin viewed through a scanning electron microscope taken before and after fractional laser treatments, as described in Example 1. FIGS. 3A and 3B show samples of skin before in vitro fractional laser treatment, while FIGS. 3C, 3D, 3E and 3F show samples of skin following in vitro fractional laser treatment. In FIG. 3A, the magnification is 100×, in FIG. 3B the magnification is 1,000×, in FIG. 3C the magnification is 100×, in FIG. 3D the magnification is 140×, in FIG. 3E the magnification is 701×, and in FIG. 3F the magnification is 3,440×.

FIG. 4 is a series of four photographs which show histological sections of skin treated under various fractional treatment parameters, as described in Example 2. The section in FIG. 4A shows skin treated in vitro using a 1550 nm laser using a 6 mJ pulse energy delivered with the contact delivery mode with a treatment zone size of 140 μm, and displays the length of a 100 μm line to indicate size. The sections in FIGS. 4B and 4C show skin treated in vitro using a 1070 nm laser with a 100 mJ pulse energy in the contact delivery mode with a treatment zone size of 140 μm, and display the length of a 200 μm line to indicate size. The section in FIG. 4D shows skin treated in vitro using a 1907 nm laser with a 12 mJ pulse energy in the non-contact delivery mode with a 140 μm treatment zone size, and displays the length of a 100 μm line to indicate size.

FIG. 5 is a series of three photographs which show histological sections obtained from human abdominal skin treated with a 1550 nm laser radiation using a 260 μm treatment zone size in the contact mode with various pulse energies, as described in Example 3. The section in FIG. 5A shows skin treated in vitro using a 15 mJ pulse energy, and displays the length of a 200 μm line to indicate the size. The section in FIG. 5B shows skin treated in vitro using a 47 mJ pulse energy, and displays the length of a 100 μm line to indicate size. The section in FIG. 5C shows skin treated in vitro using a 85 mJ pulse energy, and displays the length of a 100 μm line to indicate size.

FIG. 6 is a series of four photographs which show histological sections obtained from human abdominal skin treated in vivo using a 1550 nm laser with a pulse energy of 6 mJ delivered in both the contact and non-contact modes, as described in Example 4. The sections in FIGS. 6A and 6C show skin treated using the contact delivery mode, while the sections in FIGS. 6B and 6D show skin treated using the non-contact delivery mode. The sections in FIGS. 6A and 6B show skin excised immediately following treatment, while the sections in FIGS. 6C and 6D show skin excised one day following treatment. All sections display the length of a 100 μm line to indicate size.

FIG. 7 is a series of four photographs which show histological sections obtained from human abdominal skin treated in vivo using a 1550 nm laser with a pulse energy of 10 mJ delivered in both the contact and non-contact modes, as described in Example 4. The sections in FIGS. 7A and 7C show skin treated using the contact delivery mode, while the sections in FIGS. 7B and 7D show skin treated using the non-contact delivery mode. The sections in FIGS. 7A and 7B show skin excised immediately following the treatment, while the sections in FIGS. 7C and 7D show skin excised one day following the treatment. All sections display the length of a 100 μm line to indicate size.

FIG. 8 is a series of four photographs which show histological sections obtained from human abdominal skin treated in vivo using a 1550 nm laser with a pulse energy of 20 mJ delivered in both the contact and non-contact modes, as described in Example 4. The sections in FIGS. 8A and 8C show skin treated using the contact delivery mode, while the sections in FIGS. 8B and 8D show skin treated using the non-contact delivery mode. The sections in FIGS. 8A and 8B show skin excised immediately following the treatment, while the sections in FIGS. 8C and 8D show skin excised one day following the treatment. All sections display the length of a 100 μm line to indicate size.

FIG. 9 is a series of four photographs which show histological sections obtained from human abdominal skin treated in vivo using a 1550 nm laser with a pulse energy of 40 mJ delivered in both the contact and non-contact modes, as described in Example 4. The sections in FIGS. 9A and 9C show skin treated using the contact delivery mode, while the sections in FIGS. 9B and 9D show skin treated using the non-contact delivery mode. The sections in FIGS. 9A and 9B show skin excised immediately following the treatment, while the sections in FIGS. 9C and 9D show skin excised one day following the treatment. All sections display the length of a 100 μm line to indicate size.

FIG. 10 is a series of four photographs which show histological sections of human skin treated in vivo with fractional laser radiation, as described in Example 5. The section in FIG. 10A shows skin that has been frozen and treated with lactate dehydrogenase stain, the section in FIG. 10B shows skin that has been embedded in paraffin and stained with hematoxylin and eosin, the section in FIG. 10C shows skin that has been embedded in paraffin and treated with Gomori trichrome stain, the section in FIG. 10D shows skin that has been embedded in paraffin and treated with Fontana Masson stain. All sections display the length of a 100 μm line to indicate size.

FIG. 11 is a graph which shows the cumulative permeation of ascorbic acid over time through control skin and skin treated with fractional laser treatments as described in Example 6.

FIG. 12 is a pair of graphs which demonstrate the mean depth and width of lesions produced using the fractional laser treatments described in Example 6.

FIG. 13 is a plot of normalized cumulative ascorbic acid permeation following HPLC measurements of ex vivo skin treated with either 0 mJ (control), 10 mJ @ 2000 MTZ/cm² in contact mode, 10 mJ @ 2000 MTZ/cm² in non-contact mode, 20 mJ @ 1000 MTZ/cm² in contact mode, and 20 mJ (1000 MTZ/cm² in non-contact mode as described in Example 7. The permeation was measured at 5, 10, 15, 30, 60, and 90 minutes after treatment.

FIG. 14 is composed of four photographs, FIGS. 14A, 14B, 14C and 14 D, of ex vivo human abdominal tissue treated with the 1550 nm Fraxel® SR laser system at 10 mJ using a contact tip (14A) or non-contact tip (14B), and at 20 mJ using a contact tip (14C) or non-contact tip (14D) as described in Example 7. Paraffin embedded, H&E stained sections show the epidermal disruption when treating with the 85 μm spot size. In FIGS. 14A-14D the stratum corneum is not breached.

FIG. 15 is composed of two plots, 15A and 15B of mean lesion depth (15A), and width (15B) following treatment of human ex vivo abdominal skin at varying pulse energies using the 1550 nm Fraxel® SR laser system as described in Example 7.

FIG. 16 is a plot of cumulative 5-Fluorouracil permeation following HPLC measurements of ex vivo skin treated with either 0 mJ (control) or 10 mJ using a spot size of 60 μm and a treatment zone density of 2000 MTZ/cm² as described in Example 8.

FIG. 17 is composed of 2 photographs, FIG. 17A and FIG. 17B of ex vivo human abdominal tissue treated with the 1550 nm Fraxel Re:store™ laser system. Paraffin embedded, H&E stained sections show the epidermal disruption when treating with the 60 μm spot size. FIG. 17A shows a tissue sample taken immediately post-treatment; FIG. 17B shows a tissue sample taken 1 day post-treatment as described in Example 8.

### DETAILED DESCRIPTION

### Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3d Ed. (Plenum Press) Vols A and B(1992); Goodman and Gilman's The Pharmacological Basis of Therapeutics, Tenth Edition (McGraw-Hill, 2001); Bolognia, Jorizzo and Rapini Dermatology (Mosby, 2003); Baumann Cosmetic Dermatology: Principles and Practice (McGraw-Hill, 2002); Svelto Principles of Lasers, Fourth Edition (Springer, 2004); Siegman Lasers (University Science Books, 1986); A. J. Welch and M. J. C. van Gemert Optical-Thermal Response of Laser-Irradiated Tissue (Plenum Press, 1995).

“Ablative” refers to processes that result in the removal of a significant amount of tissue from the site of treatment, where the tissue is removed substantially instantaneously.

“Absorption coefficient” refers to a measure of the fraction of incident radiant energy absorbed per unit of thickness or mass of an absorber and can be expressed in reciprocal centimeters (cm⁻¹). For the purposes of this invention, the absorption coefficient will be understood to be expressed as the absorption coefficient of water.

“Active substance”, “active agent”, “agent”, “drug” and “substance” are used interchangeably and are intended to have their broadest interpretation as to any molecule or combination of molecules which is delivered to a living organism to produce a desired effect, such as for example a cosmetic or aesthetic effect.

“Alteration” and “altered”, as used herein when discussing events occurring within the dermis and the layers of the epidermis below the stratum corneum, are to be understood as referring events which cause significant levels of disruption within the tissue, such as formation of a vacuole, necrosis, production of necrotic debris, and/or coagulation, including thermal coagulation and photocoagulation. When discussing events occurring within the stratum corneum, “alteration” and “altered” are understood to also encompass less disruptive events, such as melting and alteration of the extra-cellular matrix, in addition to coagulation.

“Cosmetic composition” means a composition suitable for cosmetic use in a patient, including an animal or human. A cosmetic composition generally comprises an effective amount of an active substance and a cosmetic carrier. Cosmetic carriers include any of the standard cosmetic carriers, buffers and excipients, including phosphate-buffered saline solution, water, and emulsions (such as an oil/water or water/oil emulsion), and various types of wetting agents and/or adjuvants. The choice of cosmetic carriers depends upon the intended mode of administration of the active agent.

“Cosmetically effective amount” refers to the amount of an active substance sufficient to modify or improve the appearance of a physical feature, defect or irregularity when applied to the body. The cosmetic result may be cleansing, beautifying, promoting attractiveness, altering the appearance, and/or alleviating of the signs and symptoms of aging in the skin. The term “cosmetically effective amount” is used herein to denote any amount of the formulation which causes a noticeable improvement in appearance when applied to the skin once or repeatedly over a period of time in a clinically reasonable manner. The improvement can include an improvement in the appearance of the skin or of wrinkles. The amount will vary with the cosmetic result desired, the skin being treated, and the type and concentration of formulation applied. Appropriate amounts in any given instance will be readily apparent to those skilled in the art or capable of determination by routine experimentation.

“Cosmetic treatment” is a treatment administered to a patient who desires a modification or improvement in the appearance of a physical feature, defect or irregularity.

“Cross-sectional width” and “treatment zone size” are used interchangeably to describe the distance measured on the treatment zone as twice the maximum of the distance in the plane of the skin that separates each treated point in the skin from the closest viable and untreated point of the skin. In the case where a treatment zone is substantially circular, the cross-sectional width is thus equivalent to the diameter of the treatment zone.

“Dermis” refers to the layer of the skin below the epidermis that typically contains blood capillaries, blood vessels, lymph vessels, hair follicles, and various glands. The dermis is divided into the upper or papillary layer and the lower or reticular layer

“Effective amount” is a dosage sufficient to produce a desired result. The desired result may comprise a subjective or objective improvement in the patient that receives the dosage.

“Epidermis” refers to the upper or outer nonvascular layers of the skin, including the stratum corneum, stratum lucidum, stratum granulosum, stratum spinosum, and stratum basale layers.

“Local fluence”, when used in describing fractional light treatments, refers to the energy density from an optical source impacting on the surface of a tissue within the treatment zone area. Thus, the local fluence is calculated based on the energy per spot size, and can be expressed in Joules per square centimeter (J/cm²).

“Local irradiance”, when used to describe fractional light treatments, refers to the radiant power incident per unit area upon the surface of the tissue within the treatment zone area and can be expressed in Watts per square centimeter (W/cm²).

“Nonablative” and “subablative” refer to processes that do not result in significant amounts of matter being removed from the site of treatment at the time of treatment.

“Patient” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

“Permeation” is used to generally refer to the process of passing through, spreading through, or penetrating, and includes the processes of passing through cells and passing between cells.

“Pharmaceutical composition” means a composition suitable for pharmaceutical use in a patient, including an animal or human. A pharmaceutical composition generally comprises an effective amount of an active agent and a pharmaceutical carrier. Pharmaceutical carriers encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). The choice of pharmaceutical carriers depends upon the intended mode of administration of the active agent.

“Photodynamic substance”, “photodynamic active substance” and “photosensitizing agent” are used interchangeably to refer to compounds which are activated by being exposed to light. When activated by light, photodynamic substances release toxic substances, such as singlet oxygen, which can kill nearby cells, damage nearby blood vessels, and activate the immune system. Examples of photodynamic substances include psoralens, porfimer sodium, and aminolevulinic acid (ALA).

“Photodynamic therapy”, “photodynamic treatment”, “photoradiation therapy”, “phototherapy” and “photochemotherapy” are used interchangeably and refer to treatments involving administering of one or more photodynamic substances to a patient and then exposing the patient to a light source which serves to activate the photodynamic substance.

“Pore” refers to a small interstice or opening in a surface. For the purposes of this invention, a pore created in the stratum corneum by a fractional laser treatment will be understood as meaning a small opening that does not penetration through all the layers of the stratum corneum and that was created through coagulation and/or melting and retraction of the stratum corneum.

“Prophylactic treatment” is a treatment administered to a patient who does not exhibit signs of a disease or exhibits only early signs of a disease, wherein treatment is administered for the purpose of decreasing the risk of developing pathology or unwanted conditions.

“Skin” refers generally to the body's outer covering, and includes the epidermis, dermis and subcutis.

“Stratum corneum” refers to the horny outer layer of the epidermis, consisting of several layers of flat, keratinized non-nucleated dead or peeling cells, with naturally occurring pores interspersed in the tissue.

“Subcutis” and “subcutaneous tissue” are used interchangeably and refer to the layer of tissue directly under the dermis. The subcutis is composed mainly of adipose tissue, and separates the dermis from the underlying muscle.

“Substantially intact stratum corneum” refers to a stratum corneum that can have been altered by laser radiation but that remains physically present following treatment.

“Therapeutic treatment” is a treatment administered to a patient who exhibits signs of pathology, wherein treatment is administered for the purpose of diminishing or eliminating those pathological signs.

“Therapy” and “treatment” are used interchangeably and include, but are not limited to, changes in the patient's status. The changes can be either subjective or objective and can relate to features such as symptoms or signs of the disease or condition being treated. For example, if the patient notes improvements in a dermatological condition, improvements in skin appearance, reduced discomfort or decreased pain, then successful treatment has occurred. Similarly, if the clinician notes objective changes, such as by histological analysis of a biopsy sample, then treatment has also been successful. Alternatively, the clinician may note a decrease in the size of lesions or other abnormalities upon examination of the patient. This would also represent an improvement or a successful treatment. Preventing the deterioration of a patient's status is also included by the term. Therapeutic benefit includes any of a number of subjective or objective factors indicating a response of the condition being treated, or an improvement in skin appearance, as discussed herein.

“Therapeutically effective amount” refers to the amount of an active substance sufficient to induce a desired biological result. That result may be alleviation of the signs, symptoms, and/or causes of a disease, or any other desired alteration of a biological system. The term “therapeutically effective amount” is used herein to denote any amount of the formulation which causes a substantial improvement in a disease condition when applied to the affected areas once or repeatedly over a period of time in a clinically reasonable manner. The amount will vary with the condition being treated, the stage of advancement of the condition, and the type and concentration of formulation applied. Appropriate amounts in any given instance will be readily apparent to those skilled in the art or capable of determination by routine experimentation.

“Tissue” refers to an aggregate of cells that perform specific functions, including but not limited to the skin, the adipose layer located below the skin, muscle, and organs. The cells of a tissue may or may not form a layer.

“Treated region” refers to the portion of tissue which, during treatment with fractional electromagnetic radiation, is placed in the path of the device supplying the fractional laser radiation, but, by the fractional nature of the treatment, may or may not actually receive the laser radiation aimed upon it or at it.

“Treatment zone” refers to the region of tissue within a larger volume of tissue which receives an effective amount of electromagnetic radiation. Thus, when treated with fractional electromagnetic radiation, the “treated region” will contain a plurality of “discrete treatment zones” to which an effective amount of electromagnetic radiation was directed, amid one or more regions to which electromagnetic radiation was not directed.

“Treatment zone density” refers to the number of discrete treatment zones present within the surface of the treated region of skin or tissue exposed to electromagnetic radiation.

“Vacuole” refers to a small cavity or space in a tissue, including cavities or spaces that are filled with fluid or gas. As used herein, a vacuole will be understood to have a minimum cross-sectional area of 50 μm² as measured from a horizontal plane.

“Viable” refers to tissue that is composed of living cells.

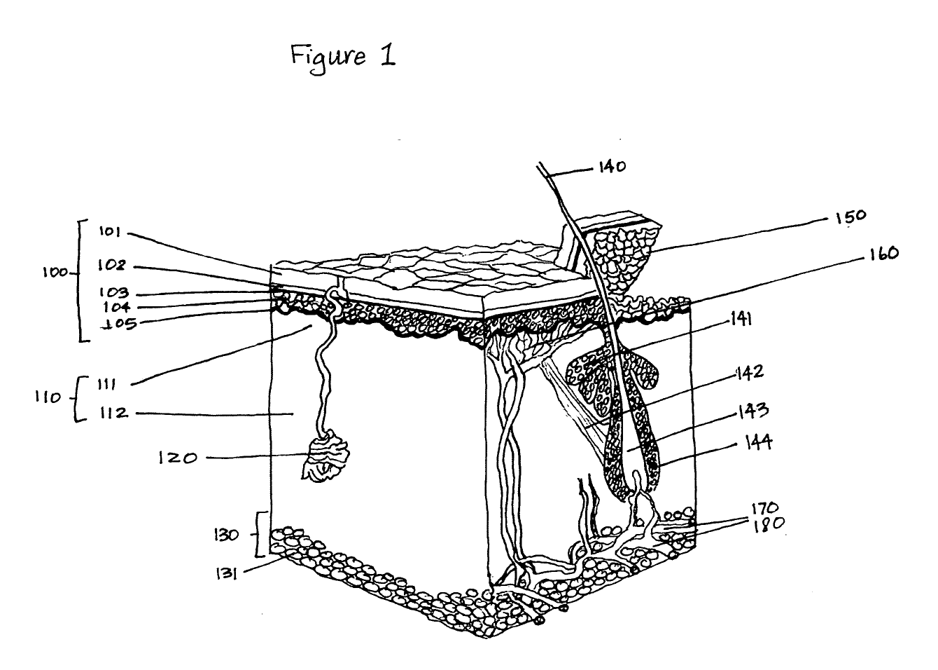

The drawing in FIG. 1 illustrates the basic structure of the skin. The skin is composed of three principal layers, the epidermis (100), dermis (110) and subcutis (130). The epidermis comprises the upper or outer layers of the skin, is nonvascular, and varies in thickness over different parts of the body. The epidermis itself is composed of several different layers, specifically the stratum corneum (101), stratum lucidum (102), stratum granulosum (103), stratum spinosum (104), and stratum basale (105) layers.

The uppermost or outermost layer of the skin is the stratum corneum (101), also known as the “horny layer” of the skin. The cells within the stratum corneum are flat and scale-like in shape. These cells, composed mainly of the protein keratin, are arranged in overlapping layers, imparting a tough and hydrophobic nature to the stratum corneum.

Below the stratum corneum (101) is the stratum lucidum (102), a homogeneous translucent band, much thinner than the layers above and below it.

Below the stratum lucidum (102) layer of the epidermis is the stratum granulosum (103), composed of two or three rows of flat cells composed mainly of keratohyalin, which is transformed into keratin in more superficial layers.

Below the stratum granulosum (103) is the stratum spinosum (104), composed of several layers of polygonal cells known as “prickle cells”. The number of layers of cells in the stratum granulosum varies over different regions of the body.

Below the stratum spinosum (104) layer is the stratum basale (105) layer, also known as the stratum germinativum, the deepest layer of the epidermis. The stratum basale is composed of columnar cells which are continually dividing to produce new skin cells. It is the cells in the stratum basale that produce melanin. Over time, the cells produced in the stratum basale move upward and away from the blood supply, and their cell contents and shapes change, forming the different layers of the epidermis. The dermal-epidermal junction is the region of the skin in which the bottom layer of the epidermis and the top layer of the dermis join.

The dermis (110) is the inner layer of the skin containing blood capillaries (160), blood vessels (170, 180), lymph vessels, hair follicles (144), and various glands, including eccrine sweat glands (120) and sebaceous glands (141). The dermis is composed of felted connective tissue containing elastin, collagen and fat. The dermis is divided into the upper, papillary layer and the lower, reticular layer.

The papillary layer of the dermis (111) typically contains a large number of dermal papillae (150), which rise perpendicularly from its surface. The papillary layer of the dermis also contains blood capillaries (160) which carry nutrients to, and remove waste from, the dividing cells in the stratum basale (105).

The reticular layer of the dermis (112) typically contains veins (170), arteries (180), sebaceous glands (141), arrector pili muscles (142), sensory nerve fibers, hair follicles (144), hair roots (143), pacinian corpuscles, hair root plexus, and eccrine sweat glands (120).

At the base of the dermis lies the subcutis (130), also known as the hypodermis or superficial fascia, composed primarily of adipose tissue (131).

The barrier properties of the stratum corneum are generally considered the main obstacles that must be overcome to allow permeation of active substances into the skin. These barrier properties can be attributed to the content and composition of the stratum corneum lipids, particularly the structural arrangement of the intercellular lipid matrix and the lipid envelope surrounding the cells. The lipids form bilayers surrounding the corneocytes, producing a “brick and mortar” model with the corneocytes as the bricks and the intercellular lipids providing the mortar.

Electromagnetic radiation, including ultraviolet radiation, visible light, infrared radiation, radar, and radio waves, can be applied directly to tissue and skin for many purposes, including for treatment of dermatological conditions, resurfacing, and to combat the effects of aging. The electromagnetic radiation can be coherent in nature, such as laser radiation, or non-coherent in nature, such as flashlamp radiation. Coherent electromagnetic radiation can be produced by lasers, including gas lasers, dye lasers, metal-vapor lasers, and/or solid-state lasers. The type of laser used with this invention can be selected from the group consisting of an argon ion gas laser, a carbon dioxide (CO₂) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, a fiber laser, an erbium doped fiber laser, a neodymium doped fiber laser, a thulium doped fiber laser, an erbium-ytterbium co-doped fiber laser, and combinations thereof. The laser can be applied in a fractional manner to produce fractional treatment. For example, the FRAXELL™ SR 1500 laser (Reliant Technologies, Inc. Mountain View, Calif.) produces fractional treatment using an erbium-doped fiber laser operating at about 1550 nm.

Treating tissue with fractional laser radiation has been found to produce fewer and less severe side effects than traditional bulk laser radiation treatments. Fractional laser radiation treatments involve the generation of a large number of discrete treatment zones within a region of tissue. The laser radiation impacts directly on only the relatively small, discrete treatment zones, instead of impacting directly on the entire region of tissue undergoing treatment, as it does in bulk treatments. Thus, a region of skin treated using a fractional laser treatment is composed of a number of discrete treatment zones where the tissue has been altered by the laser radiation, contained within a larger volume of tissue that has not been altered by the laser radiation. For both fractional treatment methods and bulk treatment methods, the tissue alterations caused by the laser radiation can take the form of thermal alterations, thermoacoustic alterations, thermomechanical alterations, and/or photomechanical alterations.

Fractional treatment methods make it possible to leave a substantial volume of tissue present within the treatment region which has not been altered by the laser radiation. When adequate amounts of viable tissue remain surrounding the discrete treatment zones following treatment, the viable tissue is able to assist in the rapid recovery of the discrete treatment zones, reducing the side effects of the laser irradiation within the region of tissue that was treated, and increasing the rate of recovery of the discrete treatment zones by stimulating skin remodeling and wound repair mechanisms. Fractional laser radiation treatments performed according to this invention maintain a substantially intact stratum corneum while producing alterations within the epidermis and dermis, so that the stratum corneum remains physically in place to provide at least some of its barrier properties, such as, for example, protection from infection.

The laser treatments of the present invention produce a plurality of individual treatment zones, increase the permeability of the treated region of skin to at least one active substance, and produce a minimal level of disruption to skin barrier function in the treated region of skin. The treatments reversibly increase permeability by producing alterations in the epidermis and dermis including a coagulation zone and a vacuole in the layers of the epidermis and dermis below the stratum corneum. The alterations can further include a plurality of pores in the stratum corneum which extend to a depth less than the full thickness of the stratum corneum.

The treatments maintain a substantially intact stratum corneum in the treated region of skin such that the treated region maintains a level of barrier function immediately after treatment equivalent to a substantial portion of the level of barrier function present in normal untreated skin. The substantial portion of the level of barrier function can be at least 60% of the level barrier function present in normal, untreated skin. The substantial portion of the level of barrier function can be at least 75% of the level barrier function present in normal, untreated skin. The substantial portion of the level of barrier function can be at least 90% of the level barrier function present in normal, untreated skin.

The level of barrier function can be determined based on an indicator of skin barrier function. The indicator of skin barrier function can be measurement of transepidermal water loss. The indicator of barrier function can be measurement of skin electrical resistance. The indicator of skin barrier function can be measurement of skin susceptibility to an irritant. The indicator of skin barrier function can be measurement of skin susceptibility to an infectious agent.

The irritant used to determine susceptibility can be a chemical irritant commonly used as a standard test agent. The standard test agent can be selected from the group consisting of 10% sodium lauryl sulphate, 1% sodium hydroxide, 30% lactic acid, and undiluted toluene.

The infectious agent used to determine susceptibility can be an exogenous pathogen. The infectious agent can be a virus. The infectious agent can be a type and strain of microbe commonly used for microbial challenge testing. The microbe can be selected from the group consisting of Candida albicans, Staphylococcus aureus, Escherichia coli, Pseudomonas aeruginosa, Bacillus subtilus, Burkholderia cepacia, and Aspergillus niger.

In one aspect of the invention, the fractional laser treatment produces an increase in the permeability of the skin to an active substance. The increase in the permeability of the skin can be reversible. The increase in the permeability of the skin can be for a duration of about 1 minute to about 5 days. The increase in the permeability of the skin can be for a duration of about 1 hour, or about 2 hours, or about 6 hours, or about 12 hours, or about 1 day, about 2 days, or about 5 days. The increase in the permeability of the skin can be for a predetermined duration based on the laser treatment parameters selected.

The fractional laser treatment methods of the invention increase the permeability of the skin while maintaining a substantially intact stratum corneum and producing alterations within the epidermis and dermis. This is in contrast to ablative laser treatments that increase skin permeability by ablating all or a substantial portion of the stratum corneum, and so do not maintain a substantially intact stratum corneum. Previous ablative treatment methods produce an increase in skin permeability that persists until the ablated portion of the skin is replaced.

The fractional laser treatment methods of the invention which increase the permeability of the skin while maintaining a substantially intact stratum corneum and producing alterations within the epidermis and dermis can also be contrasted with nonablative laser treatment methods which produce reversible increases in skin permeability in a more superficial manner by altering only the stratum corneum or only the stratum corneum and a portion of the epidermis. Previous nonablative methods of increasing skin permeability do not alter the epidermis and dermis as deep as the dermal-epidermal junction, the papillary dermis, and/or the reticular dermis.

The fractional laser treatment methods of the invention increase the permeability of the skin while maintaining a substantially intact stratum corneum and producing alterations within the epidermis and dermis can also be contrasted with nonablative laser treatment methods which do not disrupt the epidermis and dermis to as great an extent. Previous nonablative methods of increasing skin permeability do not produce as significant levels of tissue disruption in the epidermis and dermis, such as coagulation, necrosis, pores in the stratum corneum, and/or vacuoles in the layers of the epidermis below the stratum corneum.

When treating skin with the fractional laser treatment methods described herein, a wide range of treatment effects within the skin can be achieved by varying the laser treatment parameters. For example, it is possible to produce different degrees of alteration, including thermal alteration, within the epidermis and/or dermis, to produce different depths of alteration, including thermal alteration, within the epidermis and/or dermis, to produce coagulation and/or necrosis in the epidermis and/or dermis, to produce pores within the stratum corneum, and/or to produce vacuoles within the layers of the epidermis below the stratum corneum.

In another aspect of the invention, the permeability of the skin can be increased using fractional laser treatments which maintain a substantially intact stratum corneum and coagulate tissue in the epidermis and/or dermis. Specifically, depending upon the laser treatment parameters used with the fractional laser treatments described herein, the tissue below the spot where the laser radiation impinges upon the skin can be coagulated. The depth to which the region of coagulated tissue penetrates into the epidermis and dermis can be predetermined by selecting the laser treatment parameters. The region of coagulated tissue can penetrate into the papillary dermis, into the reticular dermis, or into the subcutis. The region of coagulated tissue can be between about 0.5 mm and about 4 mm below the surface of the skin.

In another aspect of the invention, the permeability of the skin can be increased using fractional laser treatments which maintain a substantially intact stratum corneum and necrose tissue in the epidermis and/or dermis. Specifically, depending upon the laser treatment parameters used with the fractional laser treatments described herein, necrosis can be produced in the tissue below the spot where the laser radiation impinges upon the skin, and the level of necrosis produced in the tissue and the depth to which the region of necrosis will penetrate into the epidermis and dermis can be predetermined by selecting the laser treatment parameters. The tissue can be treated so that between about 1% and about 10% of the cells, between about 10% and about 50%, or between about 50% and about 100% of the cells exposed to laser radiation are necrosed. Alternatively, the tissue can be treated so that between about 1% and about 10%, between about 10% and about 50%, or between about 50% and about 100% of the cells exposed to the laser radiation remain viable one day following treatment. The region of necrosed tissue can penetrate into the papillary dermis, into the reticular dermis, or into the subcutis. The region of necrosed tissue can extend between about 0.5 mm and about 4 mm below the surface of the skin.

In another aspect of the invention, the permeability of the skin can be increased using fractional laser treatments which maintain a substantially intact stratum corneum and produce pores in the stratum corneum. Specifically, these fractional laser treatments affect the surface of the stratum corneum in such a manner so as to create a plurality of discrete pores within the top layers of the stratum corneum. Without being bound by theory, the pores appear to have been formed by the coagulation, melting and/or retraction of a portion of the stratum corneum. These pores are larger in size than the naturally occurring pores, but are still small in relation to the size of the treatment zones used to create them, as they are typically between about 0.5 μm and 50 μm in diameter. Unlike naturally occurring pores, the pores created by the fractional laser treatment are superficial; they do not penetrate all the layers of the stratum corneum and do not create a direct channel to the deeper layer of the epidermis. Thus, following these fractional laser treatments, the stratum corneum remains substantially intact and in place, and maintains much of its ability to protect the body from infection.

The number and density of pores can be predetermined by selecting the laser treatment parameters. Depending upon the treatment parameters selected, the density of pores created by these fractional laser treatments can range between about 1 and about 20 pores per treatment zone, between about 20 and about 50 pores per treatment zone, or between about 50 and about 100 pores per treatment zone. Alternatively, depending upon the treatment parameters selected, the density of the pores created by these fractional laser treatments can range between about 1 and about 20 pores per 100 μm² of treated skin, between about 20 and about 50 pores per 100 μm² of treated skin, or between about 50 and about 100 pores per 100 μm² of treated skin.

In another aspect of the invention, the permeability of the skin can be increased using fractional laser treatments which maintain a substantially intact stratum corneum and produce vacuoles in the epidermis below the stratum corneum layer. Specifically, these fractional laser treatments have been found to alter the epidermis and produce a plurality of discrete vacuoles within the layers of the epidermis below the stratum corneum layer. When skin has been treated using these fractional laser treatments which produce vacuoles, the stratum corneum overlying the vacuole, although altered by the treatment, is substantially intact, a vacuole is present in the layers of the epidermis below the stratum corneum, and a region of dermal coagulation is present below the vacuole. Treatments according to this invention can also result in the dermal-epidermal junction being exposed. One day following the treatment, the stratum corneum remains substantially intact, the dermal-epidermal junction is weakened or healing, a region of dermal coagulation remains, and a majority of the cells within the lesion created by the laser treatment have lost viability. Without being bound by theory, it is possible that thermoacoustic effects caused by the laser radiation cause the formation of the vacuoles. The thermoacoustic effects can be triggered by rapid vaporization. Thermomechanical alterations of the tissue can also be involved in the creation of the vacuoles. It is also possible that the vacuoles can be created when the local fluence is of a sufficient level to heat the tissue above the boiling point of water.

The number and volume of the vacuoles can be predetermined by selecting the laser treatment parameters. Depending upon the treatment parameters used, the number of vacuoles created by these fractional laser treatments can range between about 1 and about 10 vacuoles per 10 treatment zones, between about 3 and about 7 vacuoles per 10 treatment zones, or between about 8 and about 10 vacuoles per 10 treatment zones. Additionally, depending upon the treatment parameters used, the volumes of the vacuoles created by these fractional laser treatments can range between about 50 and about 100 μm², between about 100 and about 1000 μm², or between about 1000 and about 2000 μm² when measured in a horizontal plane.

As discussed above, when treating skin with the fractional laser treatment methods described herein, a wide range of treatment effects within the skin can be achieved by varying the laser treatment parameters. These laser treatment parameters can include, for example, absorbance coefficient, wavelength, local irradiance, local fluence, pulse energy, pulse duration, treatment zone size, treatment zone density, and combinations thereof.

The absorption coefficient of the laser treatment energy is a measure of the fraction of incident radiant energy absorbed per unit of thickness of the material which is being treated with laser radiation. When skin is the material being treated and water is used as the chromophore, it can be assumed that the skin contains approximately 70% water. As used herein, the absorption coefficient is expressed as the absorption coefficient in water. To produce fractional laser treatments that maintain a substantially intact stratum corneum while producing alterations within the dermis of the treated region of skin, the wavelength of the laser light can be chosen such that its absorption coefficient in water is selected from the group consisting of between about 4 cm⁻¹ and about 150 cm⁻¹, and between about 15 cm⁻¹ and about 120 cm⁻¹. The absorption coefficient of water at different wavelengths can be found in the literature (e.g., G. M. Hale and M. R. Querry, “Optical constants of water in the 200 nm to 200 μm wavelength region,” Appl. Opt., 12, 555-563, (1973); and D. M. Wieliczka and S. Weng and M. R. Querry, “Wedge shaped cell for highly absorbent liquids: infrared optical constants of water,” Appl. Opt., 28, 1714-1719, (1989).)

The wavelength of the laser radiation is selected based on the absorption strength of various components within the tissue and the scattering strength of the tissue. These radiation transport parameters determine where the radiation energy travels in the tissue, and serve to partially determine the spatial temperature profile in the tissue. One or more than one source of laser radiation can be used in accordance with this invention. The wavelength of the laser radiation can be chosen to target a particular chromophore, such as water, elastin, collagen, sebum, hemoglobin, melanin, keratin, or other molecules present in the tissue. The epidermis contains approximately 70% water, while the stratum corneum contains approximately 10-20% water. This difference in water content between the stratum corneum and the epidermis can be beneficially used to create laser treatments which have different effects on the stratum corneum than on the epidermis and dermis. By choosing laser radiation wavelengths that act on water as the primary or substantially only chromophore, laser treatments can be used which limit the damage done to the stratum corneum.

Depending on the desired depth of treatment and desired treatment zone size, the wavelength of the laser radiation used can be selected from the group consisting of between about 1100 nm and about 2500 nm, between about 1280 nm and about 1350 nm, between about 1400 nm and about 1500 nm, between about 1500 nm and about 1620 nm, between about 1780 nm and 2000 nm, and combinations thereof. Wavelengths longer than 1500 nm can be used if the goal is to get deep penetration with small treatment zones. The shorter wavelengths generally have higher scattering coefficients than the longer wavelengths.

For the small optical beam sizes used for fractional treatment, optical scattering can be an important consideration. High absorption and/or scattering coefficients can be used to create shallow lesions that have steeper disruption profiles, which may be used to create more disruption near the surface of the skin where the barrier to permeation is highest. Lower absorption and/or scattering coefficients can be used to create greater permeation of active substances by creating narrow channels of disrupted tissue deep into the reticular dermis and/or subcutis.

To produce fractional laser treatments that maintain a substantially intact stratum corneum while producing alterations within the epidermis and dermis of the treated region of the skin, the local irradiance value can be selected from the group consisting of between about 25 kW/cm² and about 4 MW/cm², between about 0.1 MW/cm² and about 4 MW/cm², between about 0.05 MW/cm² and about 2 MW/cm², and between about 10 kW/cm² and about 800 kW/cm². For a particular treatment or patient, the local irradiance values can be determined below which pores and/or vacuoles are not formed and above which the stratum corneum is perforated during treatment. Appropriate local irradiance values for fractional laser treatments which maintain a substantially intact stratum corneum and produce pores in the stratum corneum and/or vacuoles in the epidermis would then fall within that range. Similarly, for a particular treatment or patient, a local irradiance value can be determined which produces a desired density of pores, a desired depth of coagulation and/or necrosis within the epidermis and dermis, or which produces a desired level and/or duration of increased permeability of the skin.

The laser treatment parameter fluence is a measure of the energy density impacting on the tissue in the discrete treatment zones. To produce fractional laser treatments that maintain a substantially intact stratum corneum while producing alterations within the dermis of the treated region of skin, the local fluence value can be selected from the group consisting of between about 10 J/cm² and about 320 kJ/cm², between about 4 kJ/cm² and about 160 kJ/cm², between about 1 kJ/cm² and about 40 kJ/cm², and between about 10 J/cm² and about 1600 J/cm². For a particular treatment or patient, a local fluence value can be determined below which pores and/or vacuoles are not formed and above which the stratum corneum is ruptured during treatment. Appropriate local fluence values for treatments which maintain a substantially intact stratum corneum and produce pores in the stratum corneum and/or vacuoles in the epidermis would then fall within that range. Similarly, for a particular treatment or patient, a local fluence value can be determined which produces a desired density of pores, a desired depth of coagulation and/or necrosis within the epidermis and dermis, or which produces a desired level and/or duration of increased permeability of the skin.

The laser treatment parameter pulse energy is the energy of an individual pulse of electromagnetic radiation. To produce fractional laser treatments that maintain a substantially intact stratum corneum while producing alterations within the dermis of the treated region of skin, the pulse energy can be selected from the group consisting of between about 2 mJ and about 1 J, between about 1 mJ and about 500 mJ, and between about 0.1 mJ and about 50 mJ. For a particular treatment or patient, the pulse energies can be determined below which pores and/or vacuoles are not formed and above which the stratum corneum is ruptured during treatment. Appropriate energies for treatments which maintain a substantially intact stratum corneum and producing pores in the stratum corneum and/or vacuoles in the epidermis would then fall within that range. Similarly, for a particular treatment or patient, the pulse energy can be determined which produces a desired density of pores, a desired depth of coagulation and/or necrosis within the epidermis and dermis, or which produces a desired level and/or duration of increased permeability of the skin.

The laser treatment parameter treatment zone size is the size of the beam of electromagnetic radiation at the point when it hits the surface of the target tissue, and is measured based on the cross-sectional width or diameter of the beam. To produce fractional laser treatments that maintain a substantially intact stratum corneum while producing alterations within the dermis of the treated region of skin, the spot size can be selected from the group consisting of between about 0.5 μm and about 500 μm, between about 1 μm and about 360 μm, between about 1 μm and about 250 μm, between about 1 μm and about 180 μm, about 60 μm, and about 140 μm. For a particular treatment or patient, the treatment zone size can be determined below which pores and/or vacuoles are not formed and above which the stratum corneum is ruptured during treatment. Appropriate treatment zone sizes for treatments which maintain a substantially intact stratum corneum and produce pores in the stratum corneum and/or vacuoles in the epidermis would then fall within that range. Similarly, for a particular treatment or patient, a treatment zone size can be determined which produces a desired density of pores, a desired depth of coagulation and/or necrosis within the epidermis and dermis, or which produces a desired level and/or duration of increased permeability of the skin.

The laser treatment parameter treatment zone density is the number of discrete treatment zones that are created within the treated region of tissue. To control the increase in the permeation rate of the skin, the treatment zone density of the fractional treatment can be varied. The treatment zone density can be selected from the group consisting of between about 100 and 10,000 treatment zones per square centimeter (TZ/cm²), between about 100 and about 2000 TZ/cm², between about 100 and about 1000 TZ/cm², and between about 100 and about 500 TZ/cm² of treated region of tissue. Choosing a treatment zone size in the range of about 30 to 200 μm and a treatment zone density between about 1000 and 10,000 discrete treatment zones per square centimeter can be used to provide a treatment with fewer side effects than a treatment using larger treatment zones while producing a similar increase in skin permeability. For a particular treatment or patient, a treatment zone density can be determined which produces a desired density of pores, or a desired level and/or duration of increased permeability of the skin.

In one example, fractional laser treatments of skin which increase skin permeability while maintaining a substantially intact stratum corneum and producing alterations in the epidermis and dermis of the treated region of skin can be achieved using an absorption coefficient between about 4 cm⁻¹ and 150 cm⁻¹, a local irradiance of between about 25 kW/cm² and 4 MW/cm², and a local fluence between about 10 J/cm² and 320 kJ/cm². With these laser treatment parameters, a treatment zone size between about 0.5 μm and 500 μm, and a treatment zone density between 100 and 10,000 discrete treatment zones per cm² can be used.

In another example, fractional laser treatments of skin which increase skin permeability while maintaining a substantially intact stratum corneum and producing alterations in the epidermis and dermis of the treated region of skin can be achieved using an absorption coefficient of about 4 cm⁻¹, a local irradiance between about 0.1 MW/cm² and 4 MW/cm², a local fluence between about 4 kJ/cm² and 160 kJ/cm², a pulse energy between about 2 mJ and 1 J, a treatment zone size between about 1 μm and 180 μm, and a treatment zone density between 100 and 10,000 discrete treatment zones per cm² can be used.

In another example, fractional laser treatments of skin which increase skin permeability while maintaining a substantially intact stratum corneum and producing alterations in the epidermis and dermis of the treated region of skin can be achieved using an absorption coefficient of about 8 cm⁻¹, a local irradiance between about 0.5 MW/cm² and 2 MW/cm², a local fluence between about 1 kJ/cm² and 40 kJ/cm², a pulse energy between about 1 mJ and 500 mJ, a treatment zone size between about 1 μm and 250 μm and a treatment zone density between 100 and 10,000 discrete treatment zones per cm² can be used.

In another example, fractional laser treatments of skin which increase skin permeability while maintaining a substantially intact stratum corneum and producing alterations in the epidermis and dermis of the treated region of skin can be achieved using an absorption coefficient of about 80 cm⁻¹, a local irradiance level between about 10 kW/cm² and 800 kW/cm², a local fluence between about 10 J/cm² and 1600 J/cm², a pulse energy between about 0.1 mJ and 50 mJ, a treatment zone size between about 1 μm and 360 μm and a treatment zone density between 100 and 10,000 discrete treatment zones per cm² can be used.

The use of additional components or method steps can extend the range of laser treatment parameters that can be used to produce the fractional laser treatments described herein. For example, the fractional laser radiation can be delivered using the contact delivery mode by using a contact window placed against the skin during treatment. Contact windows may be less than 100% transparent to the treatment beam wavelength or may have an absorptive layer, such as, for example, 90-99.9% transparent. Contact windows with high or low thermal conductivity can be used. The fractional laser radiation can be delivered using the non-contact delivery mode by using non-contact windows, such as, for example, windows set at a constant height above the tissue surface, or a delivery tip where the contact window has been removed.

A substantially transparent contact window with a high thermal conductivity can be used to spread the heat created in the stratum corneum by the laser energy. Sapphire or diamond windows may be used for their high thermal conductivity and transparency to pertinent wavelengths of electromagnetic radiation. The heat spreading of a thermally conductive contact window can be effectively used to reduce the thermal heat load on the stratum corneum due to the small size of the treatment zones used for fractional treatment. For this reason, the use of a contact plate to cause this type of enhancement is particularly suited to fractional treatment, but is not required.

Additionally, contact windows with low thermal conductivity can be used. Such partially transparent and/or low thermal conducting contact windows may beneficially limit heat spreading of the treatment energy. This can reduce the required treatment energy or help to confine the treatment energy, particularly when a low power laser is used.

To further extend the range of laser treatment parameters that will produce the fractional laser treatments described herein, the contact plate can be cooled to produce a thermal gradient from the surface of the skin into the skin prior to or during laser treatment. Alternatively, a gradient can be created by cooling the skin using a cryogenic spray. Examples of appropriate cooling systems and cryogenic spray systems will be evident to those skilled in the art and can be chosen based on other aspects of the treatment system.

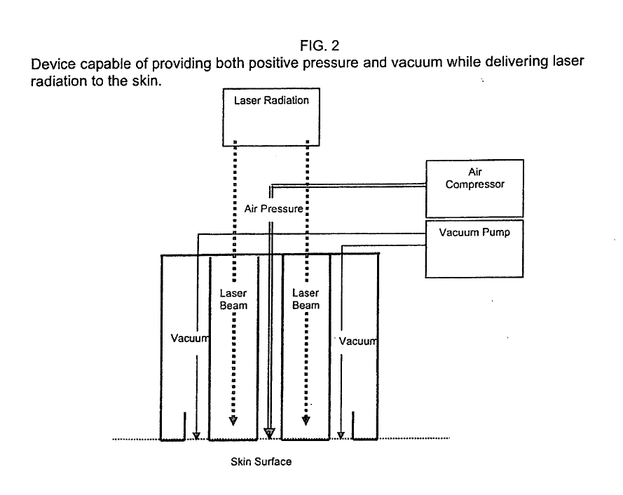

The fractional laser treatments described herein can be conducted using positive pressure to increase the rate and amount of active substance that permeates the skin. Specifically, a means for providing increased pressure in the range of about 1 to 30 pounds per square inch above atmospheric pressure can be placed against the skin before, during or after laser treatment to increase the permeability of the skin and/or the uptake of an active substance by the skin. This may be combined with a vacuum in a different location of the hand piece that can be used to hold the tip in contact with the skin. FIG. 2 is a drawing of a device capable of providing both positive pressure and vacuum while delivering laser radiation to the skin.

A model can be made for predicting the laser treatment parameters for treatments which can create vacuoles in the layers of the epidermis below the stratum corneum. The model can be made using a combined approach, wherein Monte Carlo simulations are used to model the optical propagation and absorption of incident laser light, and finite element methods are used to model heat dissipation. To enhance the accuracy of the combined model, an Arrhenius model for changes in skin constituents such as water can be added. For example, the phase change of water to steam can be approximated by including the heat of vaporization of water. Parameters for skin optics parameters, thermal conductivity of skin and contact plates, Arrhenius values, the heat of vaporization of water, and descriptions of these techniques are all publicly available and commonly known to those skilled in the art. See, e.g., A. J. Welch and M. J. C. van Gemert Optical-Thermal Response of Laser-Irradiated Tissue (Plenum Press, 1995). Further optimization of the model for determining the laser treatment parameters can be achieved without undue experimentation. As environmental factors both external and internal to the body can effect the approximations used in creating the model, some calibration should be performed on each patient prior to treatment to ensure the treatment parameters selected result in treatments within the desired ranges.

In another aspect of the invention, the fractional laser treatments described herein can be used generally to increase the permeability of the skin so as to allow the permeation of a wide range of active substances into and through the skin, where a purpose of the radiation treatment is to increase the permeability of the skin. Specifically, the fractional laser treatments described herein can be used to deliver at least one or more than one active substance to the layers of skin below the stratum corneum, for treatment of local skin or tissue conditions. These treatments can also be used to deliver active substances to the layers of skin below the stratum corneum so as to deliver active substances into the general circulation, for treatment of local or systemic conditions. The active substance can consist of one or more active substances. The active substance can be a substance that is beneficial to the patient and/or treats a condition present in the patient. The active substance can be in the form of a cosmetic composition and/or a pharmaceutical composition. The active substance can be delivered to provide a prophylactic treatment, a cosmetic treatment and/or a therapeutic treatment. The active substance can be applied once, repeatedly or continuously to the tissue before, during and/or after the laser radiation treatment.

The fractional laser treatments which produce pores and/or vacuoles that are described herein can be used for controlled delivery of active substances. Specifically, by creating pores and/or vacuoles in the skin using the fractional methods described herein and applying active substances to the fractionally treated skin, the rates at which active substances permeate through the pores and/or vacuoles and into deeper tissue and the systemic circulation can be controlled. For example, formulations containing encapsulated active substances (e.g., active substances encapsulated in liposomes, niosomes, ethosomes, transfersomes, microspheres, etc.) can be applied topically to fractionally treated skin, and the permeation of the encapsulated active substance can be controlled.

In another aspect of the invention, the fractional laser treatments described herein can be used to deliver active substances into the skin that would not permeate the skin to any measurable degree without the laser treatment. In one example, the fractional laser treatments described herein can be used to deliver a photodynamic substance into the skin, as well as to control the depth to which the photodynamic substance is applied within the skin. Following the laser treatment, the photodynamic substance can be applied to the skin, allowed to penetrate into the pores and/or vacuoles created by the laser treatment, the unpermeated portion of the substance can be removed from the surface of the skin, and the permeated portion of the substance activated by an appropriate light source. The photodynamic substance can be selected from the group consisting of a psoralen, methoxsalen, trioxsalen, 8-methoxy psoralen, porfimer sodium, aminolevulinic acid, and combinations thereof.

In another aspect of the invention, the fractional laser treatments described herein can be used similarly to other laser treatments for resurfacing, remodeling or rejuvenating skin, to treat aging of the skin, to reduce the appearance of wrinkles in the skin, and combinations thereof. In addition to producing positive laser treatment outcomes, as the fractional laser treatments described herein increase skin permeability, an active substance can be applied in conjunction with a laser treatment to increase the permeation of the active substance, increase the benefits of the laser treatment, or increase the rate of recovery from the treatment.

When the fractional laser treatments described herein are used to restore, remodel or rejuvenate skin; to treat the effects of ageing of the skin; to reduce the appearance of wrinkles in the skin and combinations thereof, an active substance can be applied in conjunction with the fractional laser treatment. The active substance can be ascorbic acid. The active substance can be selected from the group consisting of a vitamin, a mineral, an anti-oxidant, an agent that promotes skin recovery and combinations thereof. The active substance can be selected from the group consisting of a retinoid, a neurotoxin, an antibiotic, an agent for treatment of the effects of ageing of the skin, an agent for reducing the appearance of wrinkles in the skin and combinations thereof.

In another aspect of the invention, the fractional laser treatments described herein can be used similarly to other laser treatments to treat a variety of dermatological diseases and/or conditions. The dermatological disease and/or condition can include a pigmentary disorder, post-inflammatory hyperpigmentation, melasma, striae, scar tissue, and combinations thereof. When the fractional laser treatments described herein are used in this manner, an active substance can be applied in conjunction with the treatment. The active substance can be ascorbic acid. The active substance can be selected from the group consisting of a vitamin, a mineral, an anti-oxidant, an agent that promotes skin recovery, and combinations thereof. The active substance can be selected from the group consisting of a drug for treatment of a pigmentary disorder, an agent for inducing collagen remodeling, a retinoid, a neurotoxin, an antibiotic and combinations thereof.

The dermatological disease and/or condition can include acne, rosacea, alopecia, neoplasia of the skin, and combinations thereof. When the fractional laser treatments disclosed herein can be used to treat acne, rosacea and combinations thereof, an active substance can be applied in conjunction with the treatment. The active substance can be selected from the group consisting of a drug for treatment of acne, a drug for treatment of rosacea, a vitamin, a mineral, an anti-oxidant, an agent that promotes skin recovery, an antibiotic and combinations thereof.

The fractional laser treatments disclosed herein can be used to treat alopecia. When the fractional laser treatments are used in this manner, an active substance can be applied in conjunction with the treatment. The active substance can be selected from the group consisting of a drug for treatment of alopecia, a vitamin, a mineral, an anti-oxidant, an agent that promotes skin recovery, an antibiotic and combinations thereof.

The fractional laser methods disclosed herein can be used to treat a disease and/or condition selected from the group consisting of hypervascular lesions, port wine stains, capillary hemangiomas, cherry angiomas, venous lakes, poikiloderma of civate, angiokeratomas, spider angiomas, facial telangiectasias, telangiectatic leg veins, pigmented lesions, lentigines, ephelides, nevus of Ito, nevus of Ota, Hori's macules, keratoses pilaris, acne scars, epidermal nevus, Bowen's disease, actinic keratoses, actinic cheilitis, oral florid papillomatosis, seborrheic keratoses, syringomas, trichoepitheliomas, trichilemmomas, xanthelasma, apocrine hidrocystoma, verruca, adenoma sebacum, angiokeratomas, angiolymphoid hyperplasia, pearly penile papules, venous lakes, and combinations thereof. When the fractional laser treatments are used in this manner, an active substance can be applied in conjunction with the treatment. The active substance can be selected from the group consisting of a drug, a vitamin, a mineral, an anti-oxidant, an agent that promotes skin recovery, an antibiotic and combinations thereof.

The fractional laser treatments disclosed herein can be used to treat a disease and/or condition selected from the group consisting of atopic dermatitis, psoriasis, a bacterial infection, a viral infection, a fungal infection, an infestation, a neoplasm of the skin, and combinations thereof. When the fractional laser treatments are used in this manner, an active substance can be applied in conjunction with the treatment. The active substance can be selected from the group consisting of a drug for treatment of atopic dermatisis, a drug for treatment of psoriasis, an antibiotic, a drug for treatment of viral infections, a drug for treatment of fungal infections, a drug for treatment of infestations, a drug for treatment of neoplasms of the skin, a photodynamic substance, a vitamin, a mineral, an anti-oxidant, an agent that promotes skin recovery and combinations thereof.

The fractional laser treatments described herein can be used to treat other biological tissues in addition to the skin, including tissues with structures similar to human skin. For example, tissues that have an epithelium and underlying structural tissues, such the soft palate, may be treated. In addition to producing positive treatment outcomes, as the fractional laser treatments described herein increase skin permeability, an active substance can be applied in conjunction with a laser treatment to increase the permeation of the active substance, increase the benefits of the laser treatment, or to increase the rate of recovery from the treatment.

When the fractional laser treatments disclosed herein are used to deliver an active substance, the active substance can be in the form of an active substance in a carrier. The active substance can be in the form of a cosmetically effective amount of an active substance in a cosmetically acceptable carrier. The active substance can be a pharmaceutically effective amount of an active substance in a pharmaceutically acceptable carrier. The active substance can be a liquid or a semi-solid composition. The active substance can be a lotion, cream, gel or ointment. The active substance can be in the form of a paste, plaster or mask. The active substance can be in the form of a hydrogel or urethane foam infused with the active substance. The active substance can be in the form of a hydrogel or urethane mask.

When the fractional laser treatments disclosed herein are used to deliver an active substance, the active substance can be a protein or peptide. When the active substance is a protein or peptide, the protein or peptide can be naturally occurring, recombinant, or synthetic. The protein or peptide can be composed of all of the amino acids present in the naturally occurring form of the protein or peptide or can be composed of an active subset of the amino acids present in the naturally occurring protein or peptide.

The active substance that can be delivered by the fractional laser treatment methods described herein can be a local anesthetic. The local anesthetic can be selected from the group consisting of benzocaine, bupivicaine, chloroprocaine, cocaine, etidocaine, lidocaine, mepivacaine, pramoxine, prilocalne, procaine, proparacaine, ropivicaine, tetracaine, and combinations thereof.

The active substance that can be delivered by the fractional laser treatment methods described herein can be a drug for treatment of acne. The drug for treatment of acne can be selected from the group consisting of azelaic acid, benzoyl peroxide, clindamycin, erythromycin, tetracycline, trimethoprim, minicycline, doxycycline, metronidazole, sulfacetamine, sulfur, salicylic acid, a retinoid, spironolactone, cyproterone acetate, a glucocorticoid, an estrogen, a progestin, prednisone, dexamethasone, and combinations thereof.

The active substance that can be delivered by the fractional laser treatment methods described herein can be a drug for treatment of rosacea. The drug for treatment of rosacea can be selected from the group consisting of a tetracycline antibiotic, tetracycline, doxycycline, minocycline, an antibiotic, metronidazole, a beta blocker, propanolol, an antihistamine, cetirizine, loratadine, and combinations thereof.

The active substance that can be delivered by the fractional laser treatment methods described herein can be a drug to treat alopecia. The drug to treat alopecia can be selected from the group consisting of a calcium channel blocker, minoxidil, a 5-alpha reductase inhibitor, finasteride, dutasteride, a retinoid, and combinations thereof.

The drug for treatment of neoplasms of the skin can be selected from the group consisting of a retinoid, 5-fluorouracil, imiquimod, denileukin diftitox, mechlorethamine hydrochloride, carmustine, glucocorticosteroids, porfimer sodium, alpha-aminolevulinic acid, and combinations thereof.

The active substance that can be delivered by the fractional laser treatments described herein can be a photodynamic substance. The photodynamic substance can be selected from the group consisting of a psoralen, methoxsalen, trioxsalen, 8-methoxy psoralen, porfimer sodium, aminolevulinic acid, and combinations thereof.

The active substance that can be delivered by the fractional laser treatments described herein can be an antibiotic. The antibiotic can be selected from the group consisting of tetracycline, doxycycline, minocycline, erythromycin, trimethoprim, sulfamethoxazole, clindamycin, mupirocin, silver sulfadiazine, and combinations thereof.

The active substance that can be delivered by the fractional laser treatment methods described herein can be a retinoid. The retinoid can be selected from the group consisting of vitamin A, retinol, retinoic acid, tretinoin, isotreninoin, alitretionoin, etreinate, acitretin, an arotinoid, tazarotene, bexarotene, adapalene, Ro 13-7410, Ro15-1570, and combinations thereof.

The active substance that can be delivered by the fractional laser treatment methods described herein can be a neurotoxin. The neurotoxin can be selected from the group consisting of a neurotoxic compound produced by a form of Clostridia, a neurotoxic compound produced by Clostridium botulinum, a form of botulinum toxin, botulinum toxin type A, botulinum toxin type B, botulinum toxin type C, botulinum toxin type D, botulinum toxin type E, botulinum toxin type F, botulinum toxin type G, a botulinum neurotoxin peptide, a botulinum neurotoxin A (BoNT/A) peptide, a botulinum toxin in combination with a polysaccharide, a botulinum toxin in combination with a carrier comprising a polymeric backbone having attached positively charged branching groups, a botulinum toxin in combination with human serum albumin, a botulinum toxin in combination with a neuron growth inhibitor, a botulinum toxin in combination with a non-oxidizing amino acid derivative and zinc, a botulinum toxin in combination with a recombinant gelatin fragment, a stabilized botulinum toxin composition, and combinations thereof.

The active substance that can be delivered by the fractional laser treatments described herein can be a vitamin. The vitamin can be selected from the group consisting of a provitamin, a vitamin cofactor, a vitamin derivative, a form of vitamin A, a carotenoid, a retinoid, a form of B complex vitamin, thiamin, vitamin B₁, riboflavin, nicotinic acid, vitamin B₆, pyridoxine, pyridoxal, pyridoxamine, pantothenic acid, biotin, vitamin B₁₂, a form of vitamin C, ascorbic acid, a form of vitamin D, a form of vitamin E, a tocopherol, a form of vitamin K, phylloquinone, a menanquinones, a form of carnitine, choline, folic acid, inositol, and combinations thereof. The vitamin can be selected from the group consisting of a form of vitamin C, a form of vitamin A, a form of vitamin E, and combinations thereof. The active substance that can be delivered by the fractional laser treatments described herein can be a mineral. The mineral can be selected from the group consisting of a trace mineral, calcium, copper, fluoride, iodine, iron, magnesium, phosphorus, selenium, zinc, and combinations thereof.

The active substance that can be delivered by the fractional laser treatments described herein can be an anti-oxidant. The anti-oxidant can be selected from the group consisting of a vitamin, a mineral, a hormone, a carotenoid terpenoid, a non-carotenoid terpinoid, a flavonic polyphenolic, a phenolic acid, an ester of a phenolic acid, a non-flavinoid phenolic, citric acid, a lignan, a phytoestrogen, oxalic acid, phytic acid, bilirubin, uric acid, a form of lipoic acid, silymarin, a form of acetylcystine, an emblicanin antioxidant, a free-radical scavenger, a peroxiredoxin, a form of catalase, a form of superoxide dismutase (SOD), a form of glutathione, a form of thioredoxin, a form of coenzyme Q, a bioflavinoid, a green tea extract, epigallo catechin gallate (EGCG), and combinations thereof.

The active substance that can be delivered by the fractional laser treatments described herein can be an agent that promotes skin recovery. The agent that promotes skin recovery can be selected from the group consisting of an interleukin, a chemokine, a leukotriene, a cytokine, myeloperoxidase, an antibiotic, a growth factor, a heat shock protein, a matrix metalloproteinase, a hormone, an estrogen, tea tree oil, and combinations thereof.

The active substance that can be delivered by the fractional laser treatment methods described herein can be an agent for treatment of the effects of ageing of the skin. The agent for treatment of the effects of aging of the skin can be an agent for treatment of the effects of photoaging and/or chronological aging. The agent for treatment of the effects of ageing of the skin can be selected from the group consisting of a vitamin, a mineral, an antioxidant, an agent to promote recovery, a growth factor, a cytokine, a heat shock protein, an agent to induce collagen remodeling, paeoniflorin, a form of an alpha hydroxyl acid, a form of a beta hydroxyl acid, a form of kinetin, a retinoid, a form of emu oil, a form of ubiquinone, and combinations thereof. The active substance that can be delivered by the fractional laser treatment methods described herein can be an agent to reduce the appearance of wrinkles in the skin. The agent to reduce the appearance of wrinkles in the skin can be selected from the group consisting of a vitamin, a mineral, an antioxidant, an agent to promote recovery, a growth factor, a cytokine, a heat shock protein, an agent to induce collagen remodeling, a humectant, a neurotoxin, a musle relaxant, a form of an alpha hydroxyl acid, a form of a beta hydroxyl acid, an anti-oxidant, and combinations thereof.

The active substance that can be delivered by the fractional laser treatments described herein can be a growth factor. The growth factor can be naturally occurring, recombinant, or synthetic. The growth factor can be composed of all of the amino acids present in the naturally occurring form of the growth factor or can be composed of an active subset of the amino acids present in the naturally occurring growth factor. The growth factor can be selected from the group consisting of a colony stimulating factor (CSF), granulocyte-colony stimulating factor, (G-CSF), epidermal growth factor (EGF), erythropoietin (Epo), a fibroblast growth factor (FGF), FGF1, basic fibroblast growth factor, FGF2, FGF3, FGF4, a growth differentiation factor (GDF), myostatin, GDF8, GDF9, hepatocyte growth factor (HGF), an insulin-like growth factor (IGF), IGF-T, IGF-II, an interferon, INF-α, INF-β, INF-γ, leptin, nerve growth factor (NGF), a neurotropin, oncostatin (OSM), platelet-derived growth factor (PDGF), platelet growth factor (PGF), pleiotropin, thrombopoietin (TPO), a transforming growth factor (TGF), TGF-α, TGF-β, a tumor necrosis factor (TNF), TNF-α, TNF-β, vascular endothelial growth factor (VEGF), and combinations thereof. The growth factor can be selected from the group consisting of TGF-β, VEGF, EGF, leptin, a TGF, NGF, a neurotrophin, and combinations thereof.

The active substance that can be delivered by the fractional laser treatments described herein can be an agent for inducing collagen remodeling. The agent for inducing collagen remodeling can be selected from the group consisting of an endopeptidase, a zinc-dependent endopeptidase, a matrix metalloproteinase (MMP), a collagenase, a stromelysin, a matrilysin, a gelatinase, a contertase-activatable MMP, a membrane bound MMP, MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-22, MMP-23, MMP-23A, MMP-23B, MMP-24, MMP-25, MMP-26, MMP-27, MMP-28, and combinations thereof.

The active substance that can be delivered by the fractional laser treatments described herein can be a cytokine. The cytokine can be selected from the group consisting of an autocrine cytokine, an endocrine cytokine, a paracrine cytokine, and combinations thereof. The active substances that can be delivered by the fractional laser treatments described herein can be an interleukin (IL). The interleukin can be selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, and combinations thereof. The interleukin can be selected from the group consisting of IL-1, IL-2, and combinations thereof. The active substance that can be delivered by the fractional laser treatments described herein can be a heat shock protein (Hsp). Heat shock proteins, also known as stress proteins, are a group of proteins expressed when cells are exposed to elevated temperatures. The heat shock protein can be selected from the group consisting of a HspA group protein, Hsp70, Hsp71, Hsp72, Hsp78, a HspB group protein, HspB1, Hsp40, a HspC group protein Hsp90, glucose-regulated protein 94, Hsp10, Hsp20, Hsp25, Hsp27, Hsp42, Hsp60, Hsp90, Hsp100, Hsp104, Hsp110, binding immunoglobulin protein, a small heat shock protein, and combinations thereof.

The active substance that can be delivered by the fractional laser treatments described herein can be a drug for the treatment of a dermatological disease or condition. The drug for treatment of a dermatological condition can be selected from the group consisting of a drug for treatment of atopic dermatisis, a drug for treatment of psoriasis, a drug for photodynamic therapy, a drug for treatment of acne, an antibiotic, a drug for treatment of viral infections, a drug for treatment of fungal infections, a drug for treatment of infestations, a drug for treatment of neoplasms of the skin, a drug for treatment of alopecia, a drug for treatment of pigmentary disorders, and combinations thereof. The drug for treatment of pruritis can be selected from the group consisting of an antihistamine, menthol, camphor, phenol, pramoxine, doxepin, capsaicin, tar, a steroid, and combinations thereof. The drug for treatment of atopic dermatitis can be selected from the group consisting of a glucocorticosteroid, an antihistamine, a leukotriene receptor antagonist, an immunosuppressive agent, and combinations thereof. The drug for treatment of psoriasis can be selected from the group consisting of calcipotriene, anthralin, tazarotene, cytotoxic agents, acitretin, cyclosporine, mycophenolate mofetil, and combinations thereof. The drug for photodynamic therapy can be selected from the group consisting of a psoralen, methoxsalen, trioxsalen, 8-methoxy psoralen, porfimer sodium, aminolevulinic acid and combinations thereof. The drug for treatment of viral infections can be selected from the group consisting of acyclovir, famciclovir, valacyclovir, penciclovir, podophyllin, podofilox, imiquimod and combinations thereof. The drug for treatment of a fungal infection can be selected from the group consisting of an azole, fluconazole, ketaconazole, micronazole, itraconazole, econazole, econazole nitrate, an allylamine, naftifine, terbinafine, griseofulvin, ciclopirox and combinations thereof. The drug for treatment of infestations can be selected from the group consisting of gamma benzene hexachloride, lindane, premetrin, ivermectin, crotamition, sulfur, and combinations thereof. The drug for treatment of a pigmentary disorder can be selected from the group consisting of a quinolone, hydroquinolone, a corticosteroid, fluocinolone acetonide, a retinoid, a licorice extract, a bleaching agent, koijic acid, and combinations thereof.

The active substance that can be delivered by the fractional laser treatments described herein can be a drug for treatment of neoplastic diseases, including neoplastic diseases of the skin and other tissues. The drug for treatment of neoplastic diseases can be selected from the group consisting of an alkylating agent, an antimetabolite, a natural product, a hormone, a hormone antagonist, and combinations thereof. The drug for treatment of neoplastic diseases can be selected from the group consisting of a nitrogen mustard, an ethylenimine, a methylenaimine, an alkyl sulfonate, a nitrourea, a triazene, a folic acid analog, a pyrimidine analog, a purine analog, an inhibitor related to a purine analog, a vinca alkaloid, a taxane, an epipodophyllotoxin, a camptothecin, an antibiotic, an enzyme, a biological response modifier, a platinum coordination complex, an anthracenedione, a substituted urea, a methylhydralazine derivative, an adrenocortical suppressant, a tyrosine kinase inhibitor, an adrenocorticosteroid, a progestin, an estrogen, an anti-estrogen, an androgen, an anti-androgen, a gonadotropin-releasing hormone analog, and combinations thereof. The active substance that can be delivered by the fractional laser treatments described herein can be a drug for immunomodulation therapy. The drug for immunomodulation therapy can be selected from the group consisting of an immunomodulator, an immunosuppressive agent, a tolerogen, an immunostimulant, and combinations thereof. The active substance that can be delivered by the fractional laser treatments described herein can be a drug acting on the blood or on the blood-forming organs. The drug acting on the blood or on the blood-forming organs can be a hematopoietic agent, a growth factor, a mineral, a vitamin, an anticoagulant, a thrombolytic drug, an antiplatelet drug, and combinations thereof.

The active substance that can be delivered by the fractional laser treatments described herein can be a hormone, hormone agonist, or hormone antagonist. The hormone, hormone agonist or hormone antagonist can be selected from the group consisting of a pituitary hormone; a hypothalamic releasing factor; a form of thyroid; an antithyroid drug; an estrogen; a progestin; an androgen; a form of adrenalin; an adrenocorticotropic hormone; a adrenocortical steroid; a synthetic steroid analog; an inhibitor of the synthesis of an adrenocortical hormone; an inhibitor of the action of an adrenocortical hormone; insulin; an oral hypoglycemic agent; an agent affecting calcification; an agent affecting bone turnover, calcium, phosphorus, or vitamin D; calcitonin; parathyroid hormone; an analog of parathyroid hormone; an agonist of parathyroid hormone; an antagonist of parathyroid hormone; and combinations thereof. The active substance that can be delivered by the fractional laser treatment methods described herein can be a glucocorticoid. The glucocorticoid can be selected from the group consisting of betamethasone, betamethasone diproprionate, betamethasone valerate, clobetasol propionate, difluorasone diacetate, halobetasol propionate, actinomine, desoximetasone, fluocinonide, fluocinolone acetonide, flurandrenolide, hydrocortisone, hydrocortisone butyrate, hydrocortisone valerate, halcinonide, triamcinolone acetonide, amcinonide, mometasone furoate, aclometasone dipropionate, desonide, dexamethasone, dexamethasone sodium phosphate, and combinations thereof.

The active substance that can be delivered by the fractional laser treatments described herein can be an antihistamine. The antihistamine can be selected from the group consisting of doxepin hydrochloride, caribinoxamine maleate, clemastine fumarate, diphenhydramine hydrochloride, dimenhydrinate, pyrilaimine maleate, tripelennamine hydrochloride, tripelennamine citrate, chlorpheniramine maleate, brompheniramine maleate, hydroxyzine hycrochloride, hydroxyzine pamoate, cyclizine hydrochloride, cyclizine lactate, meclizine hydrochloride, promethazine hydrochloride, cyproheptadine hydrochloride, phenindamine tartrate, acrivastine, cetirizine hycrochloride, azelastine hycrocholride, lovocasastine hydrochloride, loratidine, fexofenadine, and combinations thereof.

The active substance that can be delivered the fractional laser treatments described herein can be an anti-inflammatory drug. The anti-inflammatory drug can be selected from the group consisting of histamine, a histamine antagonist, bradykinin, a bradykinin antagonist, a lipid-derived autacoid, an eicosanoid, a platelet-activating factor, an analgesic-antipyretic agent, a cyclooxygenase-2 (COX-2) inhibitor, a drug for treatment of gout, a drugs for treatment of asthma, and combinations thereof. The anti-inflammatory agent can be a non-specific COX-2 inhibitor. The non-specific COX-inhibitor can be a salicylic acid derivative, aspirin, sodium salyclate, choline magnesium trisalicylate, salsalate, diflunisal, sulfasalazine, olsalazine, a para-aminophenol derivative, acetaminophen, an indole, an indene acetic acid, indomethacin, sulindac, a heteroaryl acetic acid, tolmetrin, diclofenac, ketorolac, a arylpropionic acid, ibuprofen, naproxen, flurbiprofen, ketoprofen, fenoprofen, oxaproxin, an anthranilic acid, mefenamic acid, meclofenamic acid, an enolic acid, an oxicam, proxicam, meloxicam, an alkonone, nabumetone, and combinations thereof. The anti-inflammatory agent can be a selective COX-2 inhibitor selected from the group consisting of a diaryl-substituted furanone, a diaryl-substituted pyrazole, an indole acetic acid, a sulfonanilide, and combinations thereof. The COX-2 inhibitor can be selected from the group consisting of celecoxib; rofecoxib; meloxicam; piroxicam; valdecoxib, parecoxib, etoricoxib, CS-502, JTE-522; L-745,337; FR122047; NS398; from non-selective non-steroidal anti-inflammatory agents that would include aspirin, ibuprofen, indomethacin CAY10404, diclofenac, ketoprofen, naproxen, ketorolac, phenylbutazone, tolfenamic acid, sulindac, and others, or from steroids or corticosteroids. Compounds which selectively inhibit cyclooxygenase-2 have been described in U.S. Pat. Nos. 5,380,738, 5,344,991, 5,393,790, 5,466,823, 5,434,178, 5,474,995, 5,510,368 and WO documents WO96/06840, WO96/03388, WO96/03387, WO95/15316, WO94/15932, WO94/27980, WO95/00501, WO94/13635, WO94/20480, and WO94/26731, and are otherwise known to those of skill in the art.

The active substance that can be delivered by the fractional laser treatments described herein can be a vasoconstrictor. The vasoconstrictor can be selected from the group consisting of an antihistamine, a form of adrenaline, a form of asymmetric dimethylarinine, a form of adenosine triphosphate (ATP), a catecholamine, cocaine, a decongestant, a form of diphenhydramine, a form of endothelin, a form of phenylephrine, a form of epinephrine, a form of pseudoephedrine, a form of neuropeptide Y, a form of norepinephrine, a form of tetrahdrozoline, a form of thromboxane, and combinations thereof.

The active substance that can be delivered by the fractional laser treatments described herein can be a drug that acts at synaptic and neuroeffector junctional sites. The drug that acts at a synaptic and neuroeffector junctional site can be selected from a neurotransmitter, a muscarinic receptor agonist, a muscarinic antagonist, an anticholinerase agent, an agent acting on the neuromuscular junction and autonomic ganglia, a catecholamine, a sympathomimetic drug, an adrenergic receptor agonist, an adrenergic receptor antagonist, a serotonin receptor agonist, a serotonin receptor antagonist, and combinations thereof. The active substance that can be delivered by the fractional laser treatments described herein can be a drug that acts on the central nervous system. These drug that acts on the central nervous system can be a general anesthetic, a local anesthetic, a therapeutic gas, a hypnotic, a sedatives, a drug for treatment of depression, a drug for treatment of anxiety disorders, a drug for treatment of psychosis, a drug for treatment of mania, a drug for treatment of epilepsy, a drug for treatment of central nervous system degenerative disorders, an analgesics, an opioid analgesic, a drug for treatment of drug addiction, a drug for treatment of drug abuse, and combinations thereof. The active substance that can be delivered by the fractional laser treatments described herein can be a muscle relaxant. The muscle relaxant can be selected from the group consisting of a peripherally acting muscle relaxant, a centrally acting muscle relaxant, a directly acting muscle relaxant, a muscle relaxant acting on smooth muscle, a muscle relaxant acting on skeletal muscle, an unclassified muscle relaxant, and combinations thereof. The muscle relaxant can be selected from the group consisting of alcuronim, amyl nitrate, atacurium, baclofen, benzodiazepine, botulinum toxin, carisoprodol, chlormezanone, chlorzoxazone, cisatrcurium, curare, cyclobenzaprine, dantrolene, decamethonium, dimethyltubocurarine, doxacurium, doxacurium chloride, emylcamate, fazadinium, fazadinium bromide, febarbamate, flavoxate, fludiazepam, flunitazepam, flurazepam, gallamine, gidazepam, halazepam, hexafluoronim, loprazolam, lorazepam, lormetazepam, medazepam, mephenesin, meprobamate, metaxalone, methocarbamol, midazolam, mivacurium, mivacurium chloride, nimetazepam, nitrazepam, orphenadine, oxazepam, pancuronium, phenprobamate, phenyramidol, pinazepam, pipercuronium, pipercuronium bromide, prazepam, pridinol, quazepam, rapacuronium, rocuronium, rocuronium bromide, styramate, suxamethonium, suxamethonium chloride, tizanide, temazepam, tetrazepam, thiocolchicoside, tizanidine, tubocurarine, tolperisone, vercuronium, and combinations thereof. The active substance that can be delivered by the fractional laser treatments described herein can be a neuromuscular-blocking drug. The neuromuscular blocking drug can be selected from the group consisting of an inhibitor of acetylcholinesterase, succinylcholine, suxamethonium, decamethonium, curare, turbocurarine, atracurium, cisatacurium, vecuronium, rocuronium, mivacurium, pancuronium bromide, a form of boxulinum toxin, and combinations thereof.

The active substance that can be delivered by the fractional laser treatments described herein can be selected from the group consisting of a drug affecting renal function, a drug affecting cardiovascular function, and combinations thereof. The active substance can be selected from the group consisting of a diuretic, vasopressin, an agent affecting the renal conservation of water, renin, angiotensin, a drug for treatment of myocardial ischemia, an antihypertensive agent, a drug for treatment of hypertension, a drug for treatment of heart failure, an antiarrhythmic drug, a drug for treatment of hypercholesterolemia, a drug for treatment of dyslipidemia, and combinations thereof. The active substance that can be delivered by the fractional laser treatments described herein can be a drug affecting gastrointestinal function. The drug affecting gastrointestinal function can be selected from the group of a drug for control of gastric acidity, a drug for treatment of peptic ulcers, a drug for treatment of gastrointestinal reflux, a prokinetic agent, an antiemetic, a drug for treatment of irritable bowel syndrome, a drug used to treat diarrhea, a drug used to treat constipation, a drug used to treat inflammatory bowel disease, a drug for treatment of biliary disease, a drug for treatment of pancreatic disease, and combinations thereof. The active substance that can be delivered by the fractional laser treatments described herein can be a drug for treatment of urogenital disorders or sexual dysfunction.

The active substance that can be delivered by the fractional laser treatments described herein can be a drug for treatment of parasitic infections. The drug for treatment of parasitic infections can be selected from the group consisting of a drug for treatment of protozoal infections, a drug for treatment of malaria, a drug for treatment of amebiasis, a drug for treatment of giardiasis, a drug for treatment of trichomoniasis, a drug for treatment of trypanosomiasis, a drug for treatment of leishmaniasis, a drug for treatment of helminthiasis, and combinations thereof. The active substance that can be delivered by the fractional laser treatments described herein can be a drug for treatment of microbial diseases. The drug for treatment of microbial diseases can be selected from the group consisting of a sulfonamide, trimethoprim-sulfamethoxazole, a quinolone, a drug for treatment of urinary tract infections, a penicillin, a cephalosporin, a β-lactam antibiotic, an aminoglycoside, a protein synthesis inhibitor, an antibacterial agent, a drug for treatment of tuberculosis, a drug for treatment of Mycobacterium avium complex disease, a drug for treatment of leprosy, an antifungal agent, an antiviral agent, an antiretroviral agent, and combinations thereof. The drug for treatment of bacterial infections can be selected from the group consisting of tetracycline, doxycycline, minocycline, erythromycin, trimethoprim, sulfamethoxazole, clindamycin, mupirocin, silver sulfadiazine, and combinations thereof.

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

All publications, patents and patent applications cited herein, whether supra or infra, are hereby incorporated by reference in their entirety.

### EXAMPLES

### Example 1

### Comparison of the Surface of Treated and Untreated Skin

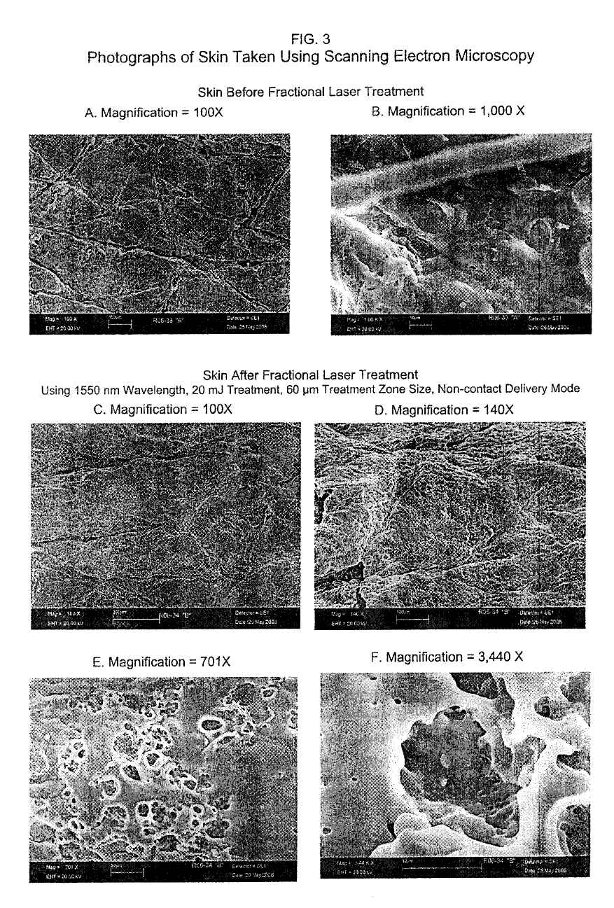

In this example, a series of photographs of the outer surface of human skin were made using a scanning electron microscope. In FIGS. 3A-3F, the skin is shown before and after fractional laser treatment to show the pores produced in the stratum corneum by the fractional laser treatment. FIGS. 3A and 3B show, at different levels of magnification, skin that has not been treated with fractional laser radiation. The normal structure of the stratum corneum is visible, including naturally occurring pores and the normal flaking of the top layer of the stratum corneum. FIGS. 3C-3F show, at four levels of magnification, skin that has been treated in vitro with fractional laser radiation using a 1550 nm laser, a pulse energy of 20 mJ, a 60 μm treatment zone size, and the non-contact delivery mode. The naturally occurring pores and the normal flaking of the top layer of the stratum corneum remain visible. A large number of pores created by the laser treatment are also visible. FIGS. 3E and 3F show that the pores created by the laser treatment do not penetrate through all the layers of the stratum corneum. FIGS. 3E and 3F also show that, after fractional laser treatment, the stratum corneum material has been disrupted and appears to have coagulated, melted, and/or retracted to form the pores. The stratum corneum has not been breached and remains in place. Although a plurality of pores have been created in the stratum corneum by the fractional laser treatment, the treated skin can be seen to maintain a substantially intact stratum corneum.

### Example 2

### Comparison of Skin Treated with Different Fractional Laser Treatments

Samples of excised human skin were treated in vitro using different fractional laser treatments before sectioning and staining to compare the effects of the treatments on the layers of the skin.

Procedure: Prior to laser treatment, each skin sample was trimmed to a size of 10 mm×60 mm and heated in between saline soaked gauze pads on a digital hot plate (Cole-Parmer Instrument Co., Vernon Hills, Ill.) until the skin surface temperature reached 98±3° F. The top layer of gauze was removed and the sample was treated at predetermined laser parameters. Immediately post-treatment, each sample was cut into smaller pieces and fixed in 10% v/v neutral buffered formalin (VWR International, West Chester, Pa.) overnight, for paraffin embedding and sectioning. The sectioned samples were stained with hematoxylin and eosin (H&E) and then imaged using a DM LM/P microscope and a DFC320 digital camera (Leica Microsystem, Cambridge, UK) The skin sections

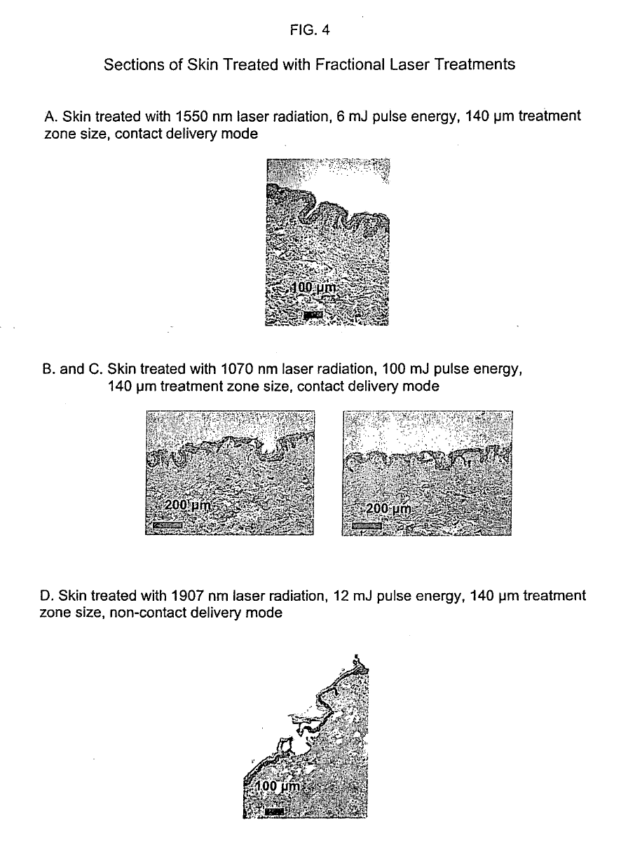

FIG. 4 is a series of photographs of histological sections of skin treated with fractional laser treatments using a variety of treatment parameters so as to show the range of alterations that can be produced within the epidermis and the dermis by the fractional laser treatments. The skin photographed in FIG. 4A is was treated with 1550 nm laser radiation using a 6 mJ pulse energy, a 140 μm treatment zone size, and the contact delivery mode. In this section, within the regions of skin below where the laser radiation impinged upon the skin, the stratum corneum remains substantially intact, a region of coagulation is visible within the epidermis and dermis, but the treatment has not created a vacuole in the epidermal layer above the region of coagulation. The skin photographed in FIGS. 4B and 4C was treated with 1070 nm laser radiation using a 100 mJ pulse energy, a 140 μm treatment zone size, and the contact delivery mode. In these two sections, within the regions of skin below where the laser radiation impinged upon the skin, the stratum corneum appears altered but remains substantially intact, and vacuoles are visible in the epidermis above a region of coagulation within the dermis. The skin photographed in FIG. 4D was treated with 1907 nm laser radiation using a 12 mJ pulse energy, a 140 μm treatment zone size, and the non-contact delivery mode. In this section, within the region of skin below where the laser radiation impinged upon the skin, a vacuole has formed within the epidermis above a region of coagulation within the dermis, but the stratum corneum above the vacuole appears to have ruptured during the treatment and so is no longer substantially intact.

### Example 3

### Comparison of Skin Treated with Fractional Laser Treatments Using the Same Wavelength of Laser Radiation and Different Pulse Energies

Samples of excised human skin were treated in vitro with fractional laser treatments using the same wavelength of laser radiation and different pulse energies before sectioning and staining to compare the effects of the treatments on the layers of the skin. The procedure described in Example 2 was used to prepare, section and stain the skin.

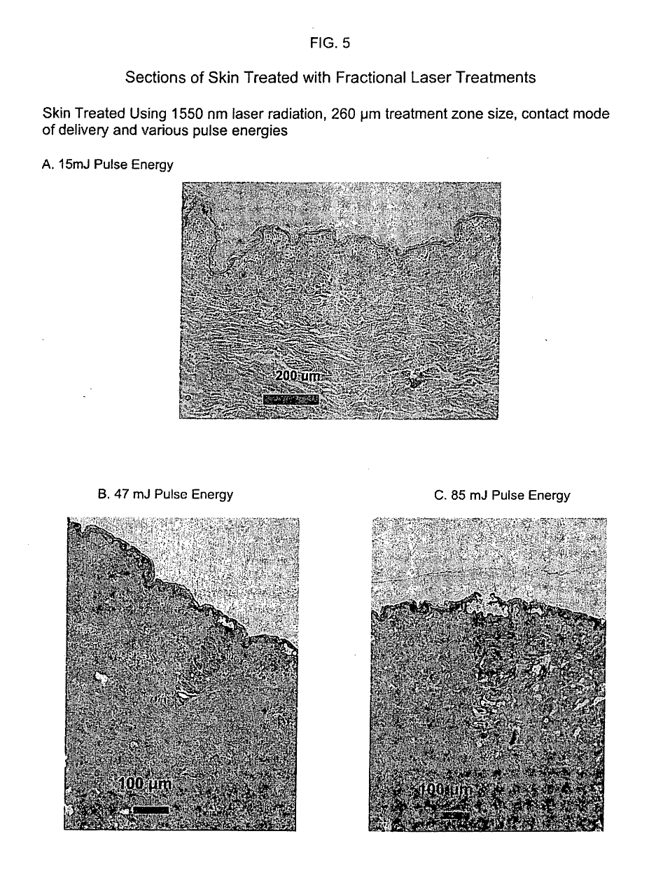

FIG. 5 is a series of photographs of histological sections of skin treated using fractional laser treatments using a range of pulse energies. The skin was treated with 1550 nm laser radiation from an erbium doped fiber laser using a 260 μm treatment zone size, the contact mode of delivery, and pulse energies of 15 mJ, 47 mJ, and 85 mJ. The skin in FIG. 5A, treated with 15 mJ, shows the treatment has produced coagulation in the epidermis and dermis but did not create a vacuole in the epidermis. The skin in FIG. 5B, treated with 47 mJ, shows the treatment has created a vacuole in the epidermis, a region of dermal coagulation below the vacuole in the dermis, and the stratum corneum overlaying the vacuole is substantially intact. The skin in FIG. 5C, treated with 85 mJ, shows the treatment has created a vacuole in the epidermis and coagulated the dermis but has ruptured the stratum corneum.

### Example 4

### Skin Treated In Vivo with Fractional Laser Radiation

Samples of human abdominal skin were treated in vivo with fractional laser radiation of various pulse energies delivered using the contact and non-contact delivery mode. The skin was excised either immediately following the treatment or one day following the treatment, sectioned, and stained to compare the effects of the radiation treatments on the layers of the skin. After the skin was excised, the sections were prepared as described in Example 2, embedded in paraffin and stained with hematoxylin and eosin.

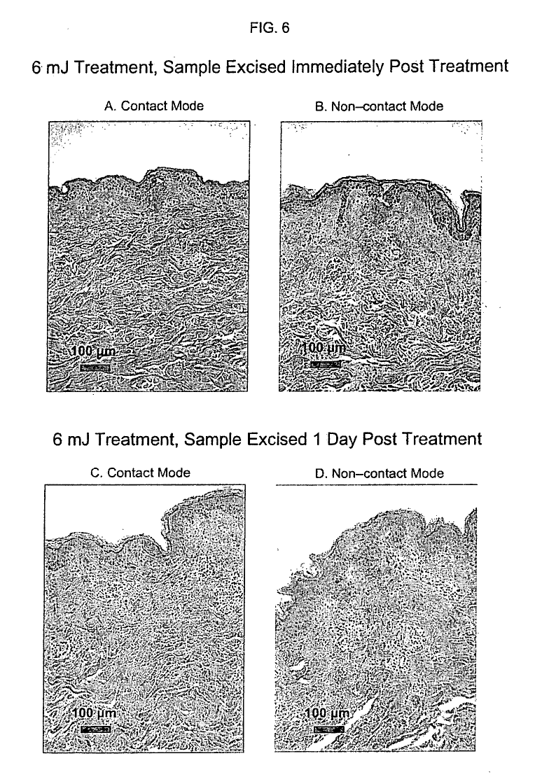

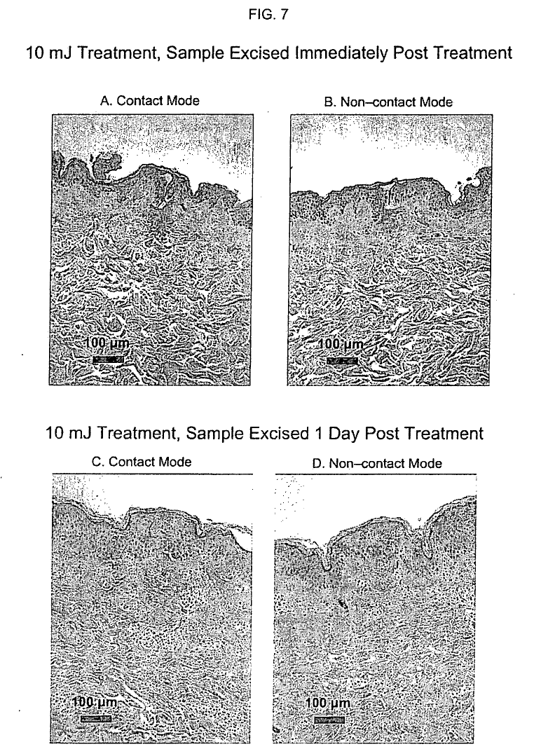

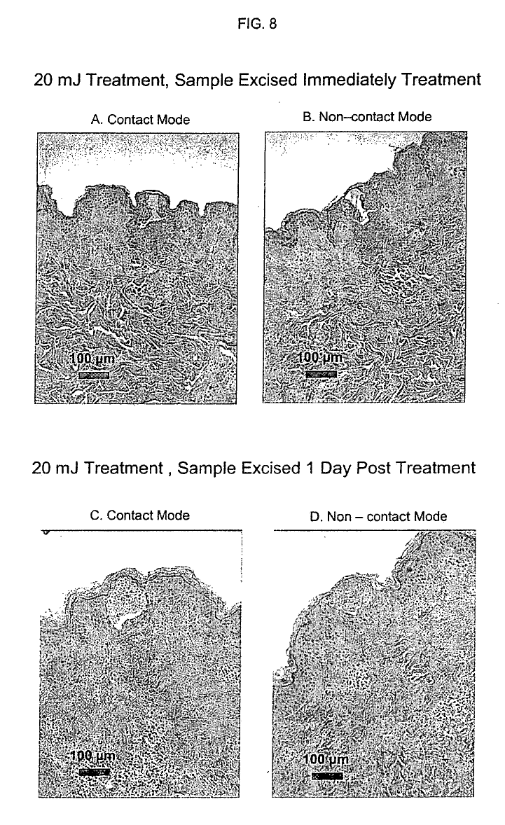

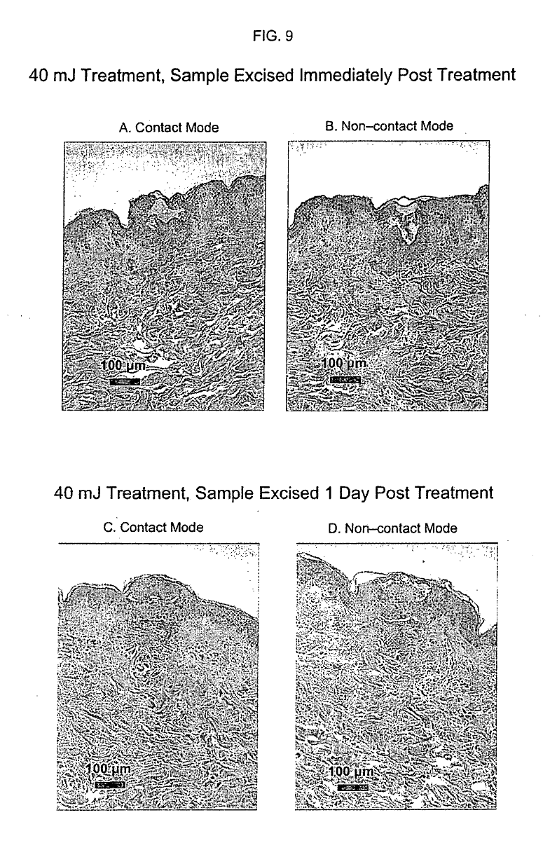

For the sections shown in FIG. 6-9, the skin was treated with a 1550 nm laser, a treatment zone size of 60 μm, and pulse energies of 6 mJ (FIG. 6), 10 mJ (FIG. 7), 20 mJ (FIG. 8) and 40 mJ (FIG. 9), using pulse durations ranging between 0.5 and 3.2 milliseconds per pulse. For FIG. 6-9, samples excised immediately post treatment are shown in sections A and B, and samples excised one day post treatment are shown in sections C and D; for sections A and C the laser radiation was delivered using a sapphire contact window (contact mode), and for sections B and D the laser radiation was delivered without a contact window (non-contact mode).

The section shown in FIG. 6A was treated using a fluence of 6 J/cm², a pulse energy of 6 mJ, a pulse duration of 0.5 milliseconds (ms), a treatment zone size of 60 μm, a treatment zone density of 1000 treatment zones per cm² (TZ/cm²) using the contact delivery mode, and was excised immediately after treatment. The section shown in FIG. 6B was treated using a fluence of 6 J/cm², a pulse energy of 6 mJ, a pulse duration of 0.5 ms, a treatment zone size of 60 μm, a treatment zone density of 1000 TZ/cm² using the non-contact delivery mode, and was excised immediately after treatment. In both these sections, a vacuole is present in the epidermis below a substantially intact stratum corneum, the dermal-epidermal junction is exposed, and a region of coagulation is present below the vacuole.

The section shown in FIG. 6C was treated using a fluence of 12 J/cm², a pulse energy of 6 mJ, a pulse duration of 0.5 ms, a treatment zone size of 60 μm, a treatment zone density of 2000 TZ/cm² using the contact delivery mode, and was excised 1 day after treatment. The section shown in FIG. 6D was treated using a fluence of 12 J/cm², a pulse energy of 6 mJ, a pulse duration of 0.5 ms, a treatment zone size of 60 μm, a treatment zone density of 2000 TZ/cm² using the non-contact delivery mode, and was excised 1 day after treatment. In both these sections, the stratum corneum overlying the vacuole remains substantially intact, the vacuole present in the epidermis appears to be filled with fluid or cellular debris, the dermal-epidermal junction appears to have healed, and the region of coagulation present below the vacuole remains visible.

The section shown in FIG. 7A was treated using a fluence of 10 J/cm², a pulse energy of 10 mJ, a pulse duration of 0.8 ms, a treatment zone size of 60 μm, a treatment zone density of 1000 TZ/cm² using the contact delivery mode, and was excised immediately after treatment. The section shown in FIG. 7B was treated using a fluence of 10 J/cm², a pulse energy of 10 mJ, a pulse duration of 0.8 ms, a treatment zone size of 60 μm, a treatment zone density of 1000 TZ/cm² using the non-contact delivery mode, and was excised immediately after treatment. In both these sections, a vacuole is present in the epidermis below a substantially intact stratum corneum, the dermal-epidermal junction is exposed, and a region of coagulation is present below the vacuole.

The section shown in FIG. 7C was treated using a fluence of 20 J/cm², a pulse energy of 10 mJ, a pulse duration of 0.8 ms, a treatment zone size of 60 μm, a treatment zone density of 2000 TZ/cm² using the contact delivery mode, and was excised 1 day after treatment. The section shown in FIG. 7D was treated using a fluence of 20 J/cm², a pulse energy of 10 mJ, a pulse duration of 0.8 ms, a treatment zone size of 60 μm, a treatment zone density of 2000 TZ/cm² using the non-contact delivery mode, and was excised 1 day after treatment. In both these sections, the stratum corneum overlying the vacuole remains substantially intact, the vacuole present in the epidermis appears to be filled with fluid or cellular debris, the dermal-epidermal junction appears to have healed or be in the process of healing, and the region of coagulation present below the vacuole remains visible.

The section shown in FIG. 8A was treated using a fluence of 20 J/cm², a pulse energy of 20 mJ, a pulse duration of 1.6 ms, a treatment zone size of 60 μm, a treatment zone density of 1000 TZ/cm² using the contact delivery mode, and was excised immediately after treatment. The section shown in FIG. 8B was treated using a fluence of 20 J/cm², a pulse energy of 20 mJ, a pulse duration of 1.6 ms, a treatment zone size of 60 μm, a treatment zone density of 1000 TZ/cm² using the non-contact delivery mode, and was excised immediately after treatment. In both these sections, a vacuole is present in the epidermis below a substantially intact stratum corneum, the vacuoles appear to be partially filled with fluid, the dermal-epidermal junction is exposed, and a region of coagulation is present below the vacuole.

The section shown in FIG. 8C was treated using a fluence of 40 J/cm², a pulse energy of 20 mJ, a pulse duration of 1.6 ms, a treatment zone size of 60 μm, a treatment zone density of 2000 TZ/cm² using the contact delivery mode, and was excised 1 day after treatment. The section shown in FIG. 8D was treated using a fluence of 40 J/cm², a pulse energy of 20 mJ, a pulse duration of 1.6 ms, a treatment zone size of 60 μm, a treatment zone density of 2000 TZ/cm² using the non-contact delivery mode, and was excised 1 day after treatment. In both these sections, the stratum corneum overlying the vacuole remains substantially intact, the vacuole present in the epidermis appears to be filled with fluid or cellular debris, the dermal-epidermal junction appears to have healed or be in the process of healing, and the region of coagulation present below the vacuole remains visible.

The section shown in FIG. 9A was treated using a fluence of 15 J/cm², a pulse energy of 40 mJ, a pulse duration of 3.2 ms, a treatment zone size of 60 μm, a treatment zone density of 375 TZ/cm² using the contact delivery mode, and was excised immediately after treatment. The section shown in FIG. 9B was treated using a fluence of 15 J/cm², a pulse energy of 40 mJ, a pulse duration of 3.2 ms, a treatment zone size of 60 μm, a treatment zone density of 375 TZ/cm² using the non-contact delivery mode, and was excised immediately after treatment. In both these sections, a vacuole is present in the epidermis below a substantially intact stratum corneum, the vacuoles appear to be partially filled with fluid, the dermal-epidermal junction is exposed, and a region of coagulation is present below the vacuole.

The section shown in FIG. 9C was treated using a fluence of 15 J/cm², a pulse energy of 40 mJ, a pulse duration of 3.2 ms, a treatment zone size of 60 μm, a treatment zone density of 375 TZ/cm² using the contact delivery mode, and was excised 1 day after treatment. The section shown in FIG. 9D was treated using a fluence of 15 J/cm², a pulse energy of 40 mJ, a pulse duration of 3.2 ms, a treatment zone size of 60 μm, a treatment zone density of 375 TZ/cm² using the non-contact delivery mode, and was excised 1 day after treatment. In both these sections, the stratum corneum overlying the vacuole remains substantially intact, the vacuole present in the epidermis appears to be filled with fluid or cellular debris, the dermal-epidermal junction appears to be in the process of healing, and the region of coagulation present below the vacuole remains visible.

### Example 5

### Skin Treated In Vivo with Fractional Laser Radiation Treated with a Variety of Stains

Human abdominal skin was treated in vivo with a 1550 nm laser, a pulse energy of 20 mJ, a treatment zone size of 60 μm, and a treatment zone density of 2000 TZ/cm². The skin was excised one day post irradiation, and paraffin and frozen sections were prepared using standard histological procedures.

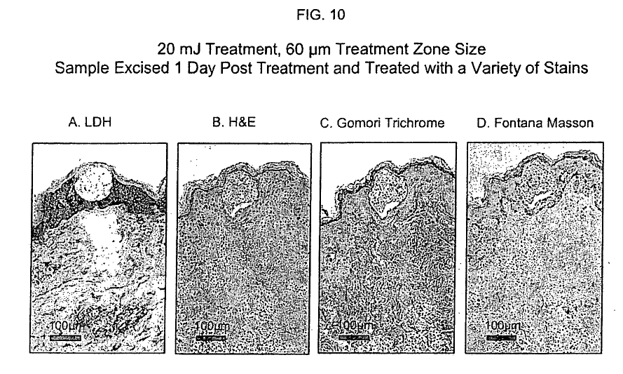

The section shown in FIG. 10A was frozen and treated with lactate dehydrogenase stain, the section shown in FIG. 10B was embedded in paraffin and stained with hematoxylin and eosin, the section shown in FIG. 10C was embedded in paraffin and treated with Gomori trichrome stain, the section shown in FIG. 10D was embedded in paraffin and treated with Fontana Masson stain. In these sections, epidermal vacuoles are present beneath a substantially intact stratum corneum and overlying regions of coagulation in the epidermis and dermis. The vacuoles appear to be filled with cellular debris, and the dermal-epidermal junction appears to be in the process of healing.

### Example 6

### Fractional Laser Treatment of Skin and Permeation of Ascorbic Acid

In vitro permeation studies were performed on untreated and treated human skin to determine if treatment of the skin with fractional laser radiation increased skin permeability to ascorbic acid. The treatment consisted of exposing the skin in vitro to fractional laser radiation. This study also evaluated the effect of using the laser radiation using the contact delivery mode and the non-contact delivery mode.

Ex vivo skin: All ex vivo laser treatments were performed using a 1550 nm laser system on freshly excised human abdominal skin (Fitzpatrick skin type II).

Laser parameters: Arrays of single mode Gaussian beams of 60 μm 1/e² diameter at incidence were delivered to the surface of the skin specimen in each treatment, using contact and non-contact tips. For the contact delivery mode, the laser radiation was delivered through a sapphire window in a contact tip which abutted the specimen. For the non-contact delivery mode, the laser radiation was delivered through a contact tip without the sapphire window; thus, the laser incidence occurred at the air-tissue interface. The laser pulse energies tested for ascorbic acid uptake measurements were 10 and 20 mJ. For each treatment, 4 passes at 250 TZ/cm² were made at a constant velocity of 1.0 cm per second producing a final spot density of 1000 TZ/cm².

To ensure that the depth of alterations made in the skin by the laser treatments did not exceed the thickness of the skin samples used for the permeation studies, the dimensions of the lesions produced using the fractional laser treatments described herein were visualized by H&E staining and were characterized. The lesion dimensions were measured using a Visual Basic computer program, and mean lesion widths and depths were calculated based on measurements of 20-25 discrete treatment zones for each treatment parameter.

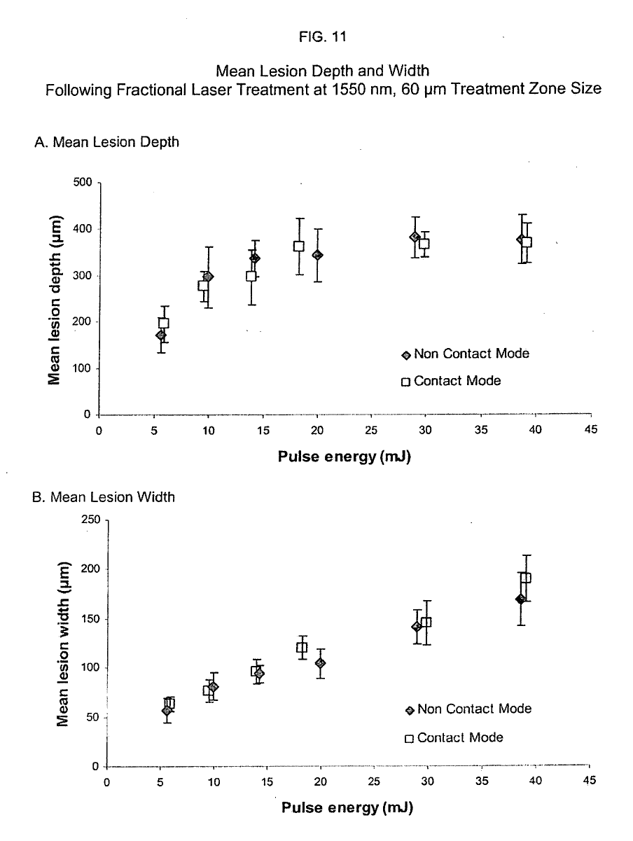

FIG. 11 represent lesion depth (FIG. 11A) and width (FIG. 11B) plots respectively. For treatments made using the contact and non-contact delivery modes, a linear increase in width and a non-linear increase in depth were observed using pulse energies in the range of 5 to 40 mJ. There was no statistically significant difference in treatment zone dimensions when comparing treatments made using the contact and the non-contact delivery mode (p>0.1). At 10 mJ, both delivery modes resulted in 300 μm deep and 80 μm wide treatment zones, while treatment zones at 20 mJ measured 350 μm deep and 110 μm wide.

Ascorbic acid formulation: Topical vitamin C solution contained L-ascorbic acid 15% (VWR International, West Chester, Pa.), ferulic acid 0.5%, and vitamin E 1% buffered to a pH of 3.2±0.2 with triethanolamine (Lin et al, 2005). Ascorbic acid was freshly prepared avoiding light exposure just prior to each experiment.

Ascorbic acid permeation studies: The uptake studies were carried out using skin permeation systems (LGA, Inc., Berkeley, Calif.) and 500 μm thick skin grafts from freshly excised human abdominal skin. Non-laser treated skin was used as a control. Immediately after laser treatment, each skin sample was mounted on a permeation system whose donor compartment was then filled with ascorbic acid solution (Lee et al, 2003). The entire arrangement was incubated to simulate body temperature. Aliquots were drawn at 5, 10, 15, 30, 60, and 90 min, and quantitatively analyzed for permeated ascorbic acid using high performance liquid chromatography (HPLC). After 90 min, each skin sample was washed thoroughly in saline, weighed, homogenized and centrifuged (Lee et al, 2003). This was followed by HPLC analysis to determine the retained ascorbic acid value. The measured retention was then normalized to the effective area of skin sample through which permeation occurred. Each experiment constituted a total of 5 individual runs (n=5).

Data analysis: The total uptake was taken as the sum of the permeated and retained ascorbic acid. The permeation values were calculated at each time point and plotted as a cumulative value. The uptake enhancement ratio represents the total ascorbic acid uptake for laser treated skin divided by the total uptake for untreated skin at 90 min.

Effect of contact mode laser treatment on ascorbic acid permeation: To assess the effect of the fractional laser treatment on ascorbic acid permeation, a 60 μm incidence treatment zone size was chosen to treat ex vivo human abdominal skin. Gross inspection of the skin post-laser treatment demonstrated no obvious structural changes.

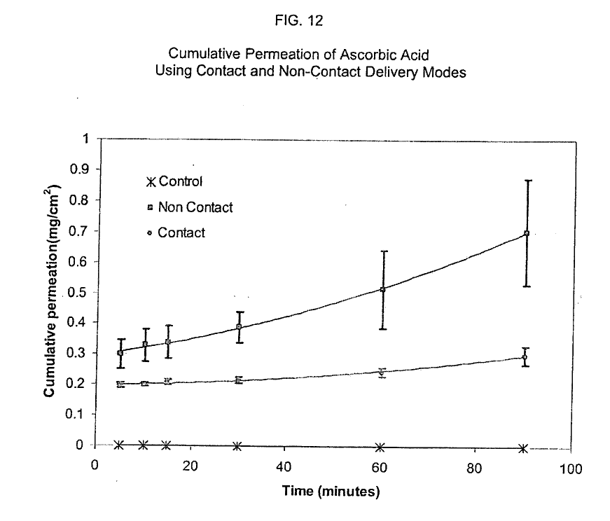

FIG. 12 shows the cumulative permeation of ascorbic acid over time through untreated (control) skin and skin treated with fractional laser energy using the contact delivery mode (using a tip with a contact window) and the non-contact delivery mode (using a tip without a contact window). Treatments made using the non-contact delivery mode at 20 mJ demonstrate higher permeation levels than treatments made at 20 mJ using the contact delivery mode.

TABLE 1
Retention and Uptake Values for Contact and Non-contact Delivery Modes

| | | | | | Enhancement ratio |
|---|---|---|---|---|---|
| Pulse | | Cumulative | | | (total uptake by |
| Energy | Treatment | permeation | Retention | Total uptake | treatment/total uptake |
| (mJ) | mode | (mg) | (mg) | (mg/cm2) | in control |
| Control | — | below | 0.06 ± 0.01 | 0.08 ± 0.01 | — |
| | | detection | | | |
| 20 | Non-contact | 0.37 ± 0.14 | 0.24 ± 0.03 | 0.77 ± 0.18 | 9.6 |
| 20 | Contact | 0.20 ± 0.03 | 0.13 ± 0.01 | 0.42 ± 0.06 | 5.3 |

Retention and Uptake Ratios for Contact and Non-contact Delivery Modes

Pulse Energy: 20 mJ
Treatment Zone Density: 1000 TZ/cm2

| | Contact | Non-contact |
|---|---|---|
| Retention Ratio | 2.1 | 3.8 |
| Total Uptake Ratio | 5.3 | 9.6 |

Retention and Uptake Ratios Based on Pulse Energy and Treatment Zone Density

| Pulse Energy | | | | |
|---|---|---|---|---|
| Treatment Zone | 10 mJ | | 20 mJ | |
| Density | 1000 TZ/cm2 | 2000 TZ/cm2 | 500 TZ/cm2 | 1000 TZ/cm2 |
| Retention Ratio | 1.8 | 2.0 | 1.3 | 3.8 |
| Total Uptake Ratio | 2.9 | 4.2 | 7.0 | 9.6 |

Table 1 shows retention and uptake values for control and treated skin. Table 1A shows the cumulative permeation, retention and total uptake of ascorbic acid in mg/cm² for treated and untreated skin, as well as the enhancement ratios for the treated skin. Table 1B shows the retention ratios (milligrams of ascorbic acid retained by treated skin divided by milligrams of ascorbic acid retained by control skin) and total uptake ratios (total mg/cm² of ascorbic acid taken up by treated skin divided by total mg/cm² of ascorbic acid taken up by control skin) for skin treated using the contact and non-contact delivery modes. Table 1C shows the retention ratios and total uptake ratios for skin treated using different pulse energies and treatment zone densities.

As shown in Table 1A, for non-irradiated ex vivo skin (control), 0.06±0.01 mg of ascorbic acid was measured in the retained fraction. Approximately 30% of the total uptake was contributed by retention and 70% was contributed by permeation. The total uptake for non-laser treated skin was 0.08±0.01 mg of ascorbic acid per gram of tissue. Skin treated at pulse energy of 20 mJ in the non-contact mode demonstrated a total uptake of 0.42±0.06 mg/cm², an enhancement ratio of 5.3 over the control. Skin treated at pulse energy of 20 mJ in the contact mode demonstrated a total uptake of 0.77±0.18 mg/cm², an enhancement ratio of 9.6 over the control.

### Example 7

### Enhanced Topical Uptake of Ascorbic Acid in Nonablative Fractional Resurfaced Ex Vivo Human Skin Using an 85 μm Incidence Microbeam Spot Size

In vitro permeation studies were performed on untreated and treated human skin to determine if treatment of the skin with fractional laser radiation increased skin permeability to ascorbic acid.

A modified 1550 nm Fraxel® SR laser system was operated as previously described by Bedi et al., Lasers Surg Med, 2007 February; 39(2):145-55. Arrays of single mode Gaussian beams of 85 μm 1/e² diameter at incidence were delivered to the surface of the skin specimen in each treatment, using contact and non-contact tips. The contact tip abutted the specimen through a sapphire window through which the laser beam was irradiated. The non-contact tip omitted the sapphire window; thus, the laser incidence occurred at the air-tissue interface. The laser pulse energies tested for ascorbic acid uptake measurements were 10 and 20 mJ. For each 10 mJ treatment, 8 passes at 250 microscopic treatment zones (MTZs) per cm² were made at a constant velocity of 1.0 cm per second producing a final spot density of 2000 MTZs per cm²; for each 20 mJ treatment, 4 passes at 250 microscopic treatment zones (MTZs) per cm² were made at a constant velocity of 1.0 cm per second producing a final spot density of 1000 MTZs per cm². These pulse energy and spot density combination parameters are similar to those used in a typical clinical NFR™ treatment session. Histologic examination was performed for pulse energies ranging from 6 to 40 mJ at a fixed spot size of 85 μm. The pulse durations ranged from 0.24 to 1.6 msec per pulse.

Prior to laser treatment, each skin sample was trimmed to a size of 10 mm×60 mm and heated in between saline soaked gauze pads on a digital hot plate (Cole-Parmer Instrument Co., Vernon Hills, Ill.) until the skin surface temperature reached 98±3° F. The top layer of gauze was removed and the sample was treated at predetermined laser parameters. Immediately post-treatment, each sample was cut into smaller pieces and fixed in 10% v/v neutral buffered formalin (VWR International, West Chester, Pa.) overnight, for paraffin embedding and sectioning. The sectioned samples were stained with hematoxylin and eosin (H&E) and then imaged using a DM LM/P microscope and a DFC320 digital camera (Leica Microsystem, Cambridge, UK). The lesion dimensions were measured using a proprietary Visual Basic computer program (Reliant Technologies, Inc., Mountain View, Calif.). Mean lesion widths and depths were calculated based on measurements of 20-25 MTZs for each treatment parameter.

Topical vitamin C solution contained L-ascorbic acid 15% (VWR International, West Chester, Pa.), ferulic acid 0.5%, and vitamin E 1% buffered to a pH of 3.2±0.2 with triethanolamine (Lin et al, 2005). Ascorbic acid was freshly prepared avoiding light exposure just prior to each experiment.

The uptake studies were carried out using skin permeation systems (LGA, Inc., Berkeley, Calif.) and 500 μm thick skin grafts from freshly excised human abdominal skin. Non-laser treated skin was used as a control. Immediately after laser treatment, each skin sample was mounted on a permeation system whose donor compartment was then filled with ascorbic acid solution (Lee et al, 2003). The entire arrangement was incubated to simulate body temperature. Aliquots were drawn at 5, 10, 15, 30, 60, and 90 min from the diffusion chamber, and quantitatively analyzed for permeated ascorbic acid using high performance liquid chromatography (HPLC). After 90 min, each skin sample was washed thoroughly in saline, weighed, homogenized and centrifuged (Lee et al, 2003). This was followed by HPLC analysis to determine the retained ascorbic acid value. The measured retention was then normalized to the effective area of skin sample through which permeation occurred. Each experiment constituted a total of 5 individual runs (n=5).

The total uptake was taken as the sum of the permeated and retained ascorbic acid over the cross-sectional area of the skin through which uptake occurred. The permeation values were calculated at each time point and plotted as a cumulative value. The uptake enhancement ratio represents the total ascorbic acid uptake for laser treated skin divided by the total uptake for untreated skin at 90 min.

TABLE 2
Retention and Uptake Values for Contact and Non-contact Delivery Modes

| | | | | | Enhancement ratio |
|---|---|---|---|---|---|
| Pulse | | Cumulative | | | (total uptake in |
| Energy | Treatment | permeation | Retention | Total uptake | treated sample/total |
| (mJ) | mode | (mg) | (mg) | (mg/cm2) | uptake in control |
| Control | — | — | 0.11 ± 0.01 | 0.14 ± 0.01 | — |
| 10 | Contact | 0.49 ± 0.07 | 0.22 ± 0.02 | 0.90 ± 0.07 | 6.6 |
| 20 | Non-contact | 2.41 ± 0.44 | 0.40 ± 0.09 | 3.58 ± 0.54 | 26.3 |
| 20 | Contact | 0.27 ± 0.10 | 0.12 ± 0.02 | 0.49 ± 0.14 | 3.6 |
| 20 | Non-contact | 0.90 ± 0.27 | 0.19 ± 0.02 | 1.40 ± 0.31 | 10.3 |

To assess the effect of the modified 1550 nm Fraxel® SR1500 laser system on ascorbic acid permeation, an 85-μm incidence microbeam spot size was chosen to treat ex vivo human abdominal skin. Gross inspection of the skin post-laser treatment demonstrated no obvious structural changes.

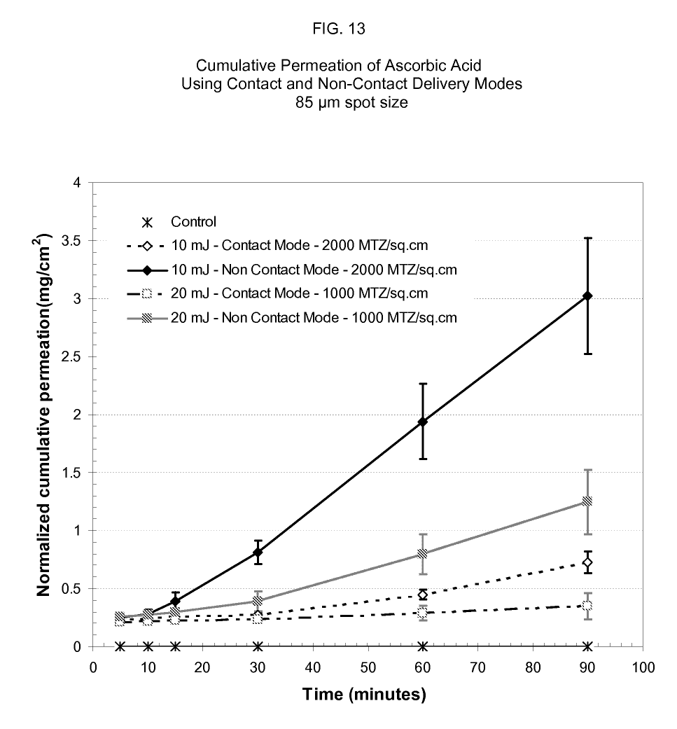

An HPLC derived standard curve for ascorbic acid values spanning 0 to 19 μg with a strong correlation coefficient was prepared (not shown), as was a normalized cumulative permeation of ascorbic acid as a function of time (FIG. 13). The ascorbic acid content of ex vivo skin prior to topical application was undetectable by HPLC. Non-irradiated ex vivo skin demonstrated no permeation up to 90 min after application as measured by HPLC.

However, under these conditions, 0.11±0.01 mg of ascorbic acid was measured in the retained fraction The total uptake of ascorbic acid at 90 min for non-laser treated skin was 0.14±0.01 mg/cm².

In sharp contrast, skin treated at pulse energies of 10 mJ and 20 mJ demonstrated permeation within 5 min (FIG. 13). By 90 min, tissue samples treated at 10 mJ @ 2000 MTZ/cm² demonstrated approximately 2× the normalized cumulative permeation to those treated at 20 mJ @ 1000 MTZ/cm². Correspondingly, a total of 0.49±0.07 mg of ascorbic acid was detected in the diffusion chamber in samples treated at 10 mJ @ 2000 MTZ/cm², and 0.27±0.10 mg of ascorbic acid was detected in samples treated at 20 mJ @ 1000 MTZ/cm² at 90 min.

There was enhanced retention in tissue samples treated at 10 mJ @ 2000 MTZ/cm² (0.22±0.02 mg, p<0.05) with respect to the control samples. Tissue samples treated with the contact mode at 20 mJ @ 1000 MTZ/cm², however, indicated statistically insignificant different retention of 0.12±0.02 mg at 90 min (p>0.05).

Total uptake of ascorbic acid was enhanced by 6.6×(0.90±0.07 mg/cm²) at 10 mJ @ 2000 MTZ/cm², and 3.6×(0.49±0.14 mg/cm²) at 20 mJ @ 1000 MTZ/cm², relative to the control.

FIG. 13 indicates that there were elevated ascorbic acid permeation at 5 min for both 10 mJ and 20 mJ in the non-contact mode. By 90 min, the normalized cumulative permeation at 10 mJ @ 2000 MTZ/cm² was more than twice that of 20 mJ @ 1000 MTZ/cm².

Table 2 shows that for tissue samples treated at 10 mJ @ 2000 MTZ/cm², 2.41±0.44 mg of ascorbic acid had permeated; correspondingly, for tissue samples treated at 20 mJ @ 1000 MTZ/cm², 0.90±0.27 mg of ascorbic acid was detected at 90 min.

There were also enhanced retention of 0.40±0.09 mg with the non-contact mode at 10 mJ @ 2000 MTZ/cm², and 0.19±0.02 mg at 20 mJ @ 1000 MTZ/cm² with respect to the control (p<0.05).

Overall, the total uptake was enhanced by 26.3×(3.58±0.54 mg/cm²) at 10 mJ @ 2000 MTZ/cm², and 10.3×(1.40±0.31 mg/cm²) at 20 mJ @ 1000 MTZ/cm² over control treatments by 90 min.

At 5 min, there was no difference in permeation between contact mode and non-contact mode regardless of pulse energies or spot densities utilized (FIG. 13). However, by 30 min, tissue samples treated in the non-contact mode exhibited a more rapid rise in the rate of permeation as compared to those in the contact mode. In addition, the normalized cumulative permeation of samples treated at 10 mJ exceeded those at 20 mJ in their respective mode, and this continued through 90 min; interestingly, the spot density employed in the 10 mJ treatments was twice that in the 20 mJ treatments.

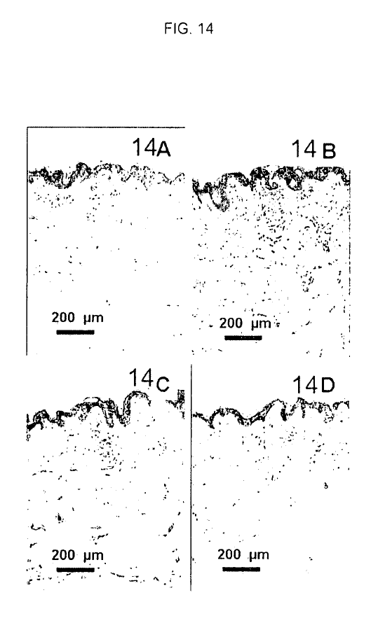

Histologic observations demonstrated that there was no alteration in the structural integrity of untreated skin exposed to ascorbic acid for 90 min (not shown). On the other hand, skin irradiated at 10 or 20 mJ in the contact mode demonstrated epidermal disruption including vacuole formation without any visible effect on the stratum corneum (FIGS. 14A and 14C, respectively). A region of dermal coagulation was observed to underlie each disrupted epidermal zone. This was also the case when switching to the non-contact mode (FIGS. 14B and 14D), with more pronounced epidermal disruption overall. These macroscopic effects appeared to intensify with increasing pulse energy as a result of higher fluence or irradiance level.

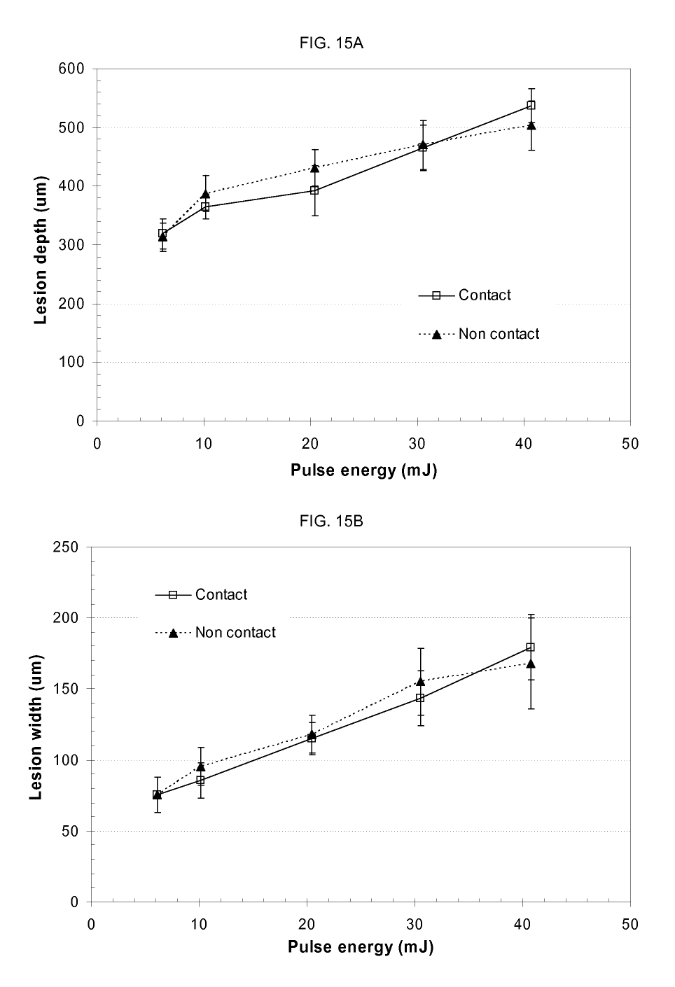

To ensure that an ex vivo skin thickness of 500 μm exceeded the depth of thermal injury induced by the range of experimental treatment parameters, we characterized the lesion dimensions obtained by H&E staining. FIGS. 15A and 15B represent lesion depth and width plots, respectively, and summarize the results of the experiments exemplified in FIGS. 14A-14D. For both contact and non-contact mode treatments, a linear increase in width (FIG. 15B) but a plateau in depth (FIG. 15A) in the pulse energy range of 5 to 40 mJ was observed. There was no statistically significant difference in lesion dimensions when comparing contact and non-contact treatments (p>0.05). At 10 mJ, both modes resulted in approximately 320-μm deep and 75-μm wide lesions, while lesions at 20 mJ measured 400 μm deep and 120 μm wide (FIGS. 15A and 15B).

In this study, an 85 μm spot size and final spot densities of 2000 MTZs and 1000 MTZs were evaluated. The ultrahigh microscopic fluences and irradiances of the treatments cause suprathreshold transient temperature and submicron thermoacoustic effects on the stratum corneum. This results in altered ultrastructure of the stratum corneum facilitating topical uptake of molecules such as, for example hydrophilic small molecules including ascorbic acid. The treatment, however, preserves the overall macroscopic stratum corneum integrity thus maintaining barrier function.

The low absorption coefficient at this wavelength preventsablation of the stratum corneum at very high microbeam fluence levels in excess of 0.5 kJ per cm². Given the short pulse durations (0.24-1.6 msec) and the small microbeam spot size (85 μm) of this laser system, the microfluence and irradiance levels were extremely high at 10 mJ (176 J per cm² and 440 kW per cm² respectively) and at 20 mJ (350 J per cm² and 440 kW per cm² respectively).

Under all conditions of laser treatment tested, ascorbic acid was observed to permeate into the diffusion chamber (Table 2).

Although no statistically significant difference in lesion dimensions was observed between treatments in the contact and non-contact mode (FIGS. 15A and 15B), the latter consistently resulted in slightly more epidermal disruption. Neither mode of treatment caused ablation of the stratum corneum, effectively maintaining barrier function against pathogen entry (FIGS. 14A-14D). In the contact mode, the sapphire window abutted with the stratum corneum acted as an acoustic impedance matching material, dampening or eliminating any thermoacoustic perturbation on the stratum corneum as a result of laser irradiation. These experiments demonstrated a 26.3× and 10.3× enhancement of ascorbic acid uptake in the non-contact mode at 10 mJ @ 2000 MTZ/cm² and 20 mJ @ 1000 MTZ/cm² over the control, respectively, and representing nearly 4× and 3× improvement over contact mode treatments at identical laser parameters.

This study confirms that the uptake of ascorbic acid across laser-treated skin is dependent on treatment zone density as well as the total number of treatment zones present in a region of skin exposed to an active substance. These results showed that regardless of pulse energy (10 mJ or 20 mJ), the total uptake through tissue samples treated at 2000 MTZ/cm² was approximately two or more times those treated at 1000 MTZ/cm² within their respective treatment mode (contact or non-contact), as final treatment zone density for a given area of skin (and thus the total number of treatment zones produced) plays a more important role than absolute pulse energy or microbeam fluence. Since lower energy setting treatments have been found to be less painful for any final treatment zone density, enhancement of ascorbic acid uptake can be achieved clinically in the absence of significant pain.

This study demonstrated significant enhancement of ascorbic acid uptake in the absence of any stratum corneum ablation or removal, unlike devices such as microdermabrasion and ablative lasers where ablation or removal is a prerequisite for efficacy. The treatment used in this study also did not involve the use of any exogenous absorber, whether superficial or delivered subcutaneously, in conjunction with laser irradiation to disrupt or alter the ultrastructure of the skin. Contact mode as well as non-contact mode treatments were observed to produce enhanced uptake due to epidermal disruption (e.g., the formation of vacuoles below the stratum corneum within the treatment zones). Nanoscale changes in the stratum corneum ultrastructure (i.e., the formation of pores in the stratum corneum which extend to a depth less than the full thickness of the stratum corneum) also contributed to increased uptake. In addition, the number and density of treatment zones was confirmed to play a significant role in increasing total uptake, even at lower pulse energies.

### Example 8

### Uptake of 5-Fluorouracil from Fractionally Treated Skin

In vitro permeation studies were performed on untreated and treated ex vivo human skin to determine if treatment of the skin with fractional laser radiation increased skin permeability to a 0.5% (w/v) 5-Fluorourocil solution. The treatment was conducted using the methods outlined in Examples 6 and 7 using a Fraxel Re:store™ laser system (Reliant Technologies, Mountain View, Calif., USA). The treatment parameters used included a laser pulse energy of 10 mJ, a 60 μm incident optical spot size, and a final spot density of 2000 MTZ/cm². The skin graft thickness used was 500 μm. The experiment consisted of a total of 6 individual runs (n=6). The uptake study was conducted using an infinite dose regimen.

TABLE 3

| | Ratio ± Standard |
|---|---|
| Measurement | Deviation |
| Permeation | 37.7 ± 10.6 mg |
| Retention | 2.4 ± 0.8 mg |
| Uptake | 20.3 ± 5.3 mg |

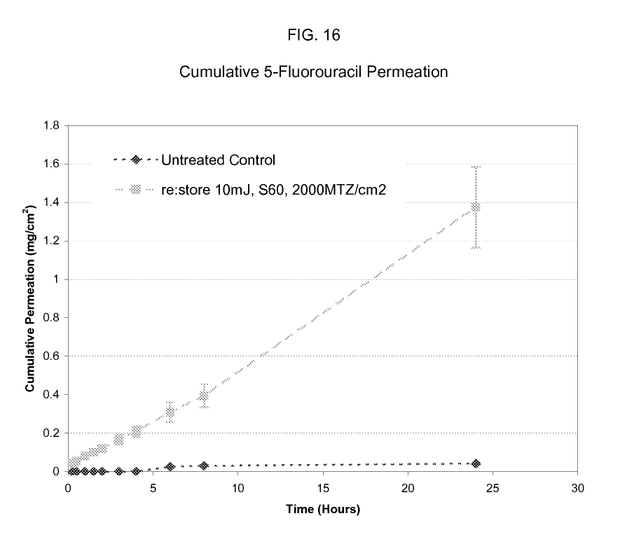

The total uptake was taken as the sum of the permeated and retained 5-Fluorouracil. The permeation values were calculated at each time point and plotted as a cumulative value in FIG. 16. Over the course of the study (24 hours), the uptake of 5-Fluorouracil from the untreated skin (control) remained below 0.2 mg/cm², while the uptake of 5-Fluorouracil from the laser treated skin approached 1.4 mg/cm² after 24 hours.

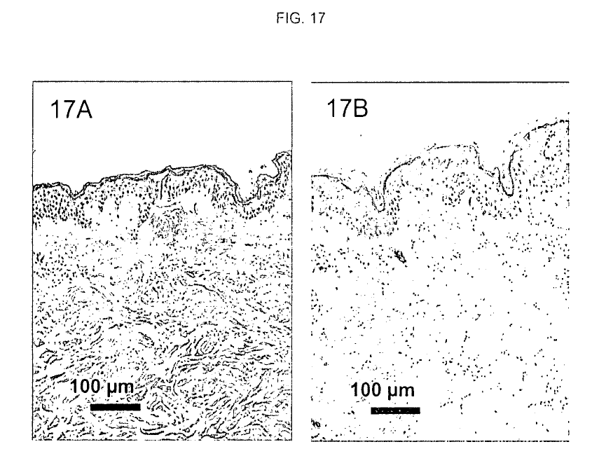

FIGS. 17A and 17B are photographs of histologic samples of skin immediately post-treatment (FIG. 17A) and skin 1 day post-treatment (FIG. 17B). After the skin was removed, it was embedded in paraffin and stained with hematoxylin and eosin. These histologic observations show that the treated skin experienced epidermal disruption including vacuole formation without any visible effect on the stratum corneum immediately post-treatment, and a region of dermal coagulation was observed to underlie the disrupted epidermal zone (FIG. 17A). The one day post-treatment sample (FIG. 17B) showed partial healing of the epidermal disruption, including a reduced region of dermal coagulation and partial filling in of the vacuole.

What is claimed is:

1. A method of increasing the permeability of skin, the method comprising:
selecting a region of skin in need an increase in permeability to an active substance and treating the region of skin with fractional laser radiation, wherein said treating produces the increase in the permeability of the skin to the active substance and maintains a substantially intact stratum corneum.

2. The method of claim 1 wherein the increase in the permeability of the skin to the active substance is reversible.

3. The method of claim 2 wherein the increase in the permeability of the skin is for a duration of about 1 hour, about 2 hours, about 6 hours, about 12 hours, about 1 day, about 2 days, or about 5 days.

4. The method of claim 1, wherein the treating produces alterations in the epidermis and dermis of the skin.

5. The method of claim 4, wherein the alterations in the epidermis and dermis consist of coagulated tissue.

6. The method of claim 4, wherein the alterations in the epidermis and dermis consist of necrosed tissue.

7. The method of claim 1, wherein the treating produces pores in the stratum corneum.

8. The method of claim 1, wherein the treating produces vacuoles in the epidermis below the stratum corneum.

9. The method of claim 1 wherein the fractional laser radiation has an absorption coefficient between about 4 cm⁻¹ and about 150 cm⁻¹, or between about 15 cm⁻¹ and about 120 cm⁻¹.

10. The method of claim 1 wherein the fractional laser radiation has a wavelength selected from the group consisting of between about 1100 nm and about 2500 nm, between about 1280 nm and about 1350 nm, between about 1400 nm and about 1500 nm, between about 1500 nm and about 1620 nm, between about 1780 nm and 2000 nm, and combinations thereof.

11. The method of claim 1 wherein the fractional laser radiation has a wavelength of 1550 nm.

12. The method of claim 1 wherein the fractional laser radiation has a local irradiance selected from the group consisting of between about 25 kW/cm² and about 4 MW/cm², between about 0.1 MW/cm² and about 4 MW/cm², between about 0.05 MW/cm² and about 2 MW/cm², and between about 10 kW/cm² and about 800 kW/cm².

13. The method of claim 1 wherein the fractional laser radiation has a local fluence selected from the group consisting of between about 10 J/cm² and about 320 kJ/cm², between about 4 kJ/cm² and about 160 kJ/cm², between about 1 kJ/cm² and about 40 kJ/cm², and between about 10 J/cm² and about 1600 J/cm².

14. The method of claim 1 wherein the fractional laser radiation has a pulse energy selected from the group consisting of between about 2 mJ and about 1 J, between about 1 mJ and about 500 mJ, and between about 0.1 mJ and about 50 mJ.

15. The method of claim 1 wherein the fractional laser radiation has a treatment zone size selected from the group consisting of between about 0.5 μm and about 500 μm, between about 1 μm and about 360 μm, between about 1 μm and about 250 μm, between about 1 μm and about 180 μm, about 60 μm, and about 140 μm.

16. The method of claim 1 wherein the fractional laser radiation has a treatment zone density selected from the group consisting of between about 100 and 10,000 TZ/cm², between about 100 and about 2000 TZ/cm², between about 100 and about 1000 TZ/cm², and between about 100 and about 500 TZ/cm².

17. The method of claim 1 wherein the fractional laser radiation is delivered using a contact window.

18. The method of claim 1 wherein the fractional laser radiation is delivered using a non-contact window.

19. The method of claim 1 wherein the region of skin is cooled during the treating.

20. The method of claim 1 wherein positive pressure is applied to the region of skin during the treating.

21. The method of claim 1 wherein a vacuum is applied to the region of skin during the treating.

22. The method of claim 1 wherein the increase in the permeability of the skin to the active substance is used to deliver the active substance.

23. The method of claim 1 wherein the increase in the permeability of the skin to the active substance is used to deliver a photodynamic active substance into the skin.

24. The method of claim 1 wherein the treating is used to restore, remodel or rejuvenate skin; to treat aging of the skin; to reduce the appearance of wrinkles in the skin; and combinations thereof.

25. The method of claim 1 the treating is used to treat a pigmentary disorder, post-inflammatory hyperpigmentation, melasma, striae, scar tissue, and combinations thereof.

26. The method of claim 1 wherein the treating is used to treat acne, rosacea, alopecia, neoplasia of the skin, and combinations thereof.

27. The method of claim 1 wherein the active substance is applied in conjunction with the fractional laser treatment.

28. The method of claim 27 wherein the active substance is applied before treatment, during treatment, after treatment, or combinations thereof.

29. The method of claim 27 wherein the active substance is applied once, repeatedly, or continuously.

30. The method of claim 27 wherein the active substance comprises a pharmaceutical composition or a cosmetic composition.

31. The method of claim 27 wherein the active substance is a local anesthetic, a drug for treatment of acne, a drug for treatment of rosacea, a drug for treatment of alopecia, a drug for treatment of neoplasia of the skin, a photodynamic substance, an antibiotic or combinations thereof.

32. The method of claim 27 wherein the active substance is a retinoid.

33. The method of claim 27 wherein the active substance is a neurotoxin.

34. The method of claim 27 wherein the active substance is selected from the group consisting of a vitamin, a mineral, an anti-oxidant, an agent that promotes skin recovery or combinations thereof.

35. The method of claim 27 wherein the active substance is vitamin C.

36. A method of increasing the permeability of skin, the method comprising:
treating a region of skin with laser radiation, wherein the treating produces a plurality of individual treatment zones,
increases the permeability of the region of skin to at least one active substance, and produces a minimal level of disruption to skin barrier function in the region of skin.

37. A method of increasing the permeability of skin, the method comprising:
treating a region of skin with laser radiation in a manner so as to produce a plurality of individual treatment zones within the region of skin, wherein said treating reversibly increases permeability of the region of skin to at least one active substance by producing alterations in the epidermis and dermis including a coagulation zone and a vacuole in the layers of the epidermis and dermis below the stratum corneum, and wherein said treating maintains a substantially intact stratum corneum in the region of skin such that the region of skin maintains a level of barrier function immediately after the treating equivalent to at least 60% of a level of barrier function present in normal untreated skin based on an indicator of skin barrier function.

38. The method of claim 37, wherein the alterations in the epidermis and dermis further comprise a plurality of pores in the stratum corneum which extend to a depth less than the full thickness of the stratum corneum.

39. The method of claim 37, wherein the indicator of skin barrier function is measurement of transepidermal water loss.

40. The method of claim 37, wherein the indicator of skin barrier function is measurement of skin electrical resistance.

41. The method of claim 37, wherein the indicator of skin barrier function is measurement of tritiated water flux.

42. The method of claim 37, wherein the indicator of skin barrier function is measurement of skin susceptibility to an irritant.

43. The method of claim 37, wherein the indicator of skin barrier function is measurement of skin susceptibility to an infectious agent.

44. The method of claim 37, wherein an increase in a level of uptake of the active substance is dependent on the number of independent treatment zones created during the treating.

45. The method of claim 37, wherein an increase in a level of uptake of the active substance is dependent on the density of independent treatment zones created during the treating.

46. A method of delivering an active substance to the skin, the method comprising the steps:
treating a region of skin with laser radiation in a manner so as to produce a plurality of individual treatment zones within the region of skin, wherein said treating increases permeability of the region of skin to at least one active substance for a limited period of time by producing alterations in the epidermis and dermis including a coagulation zone and a vacuole in the layers of the epidermis below the stratum corneum, and wherein said treating maintains a substantially intact stratum corneum in the region of skin such that the region of skin maintains a level of skin barrier function immediately after the treating equivalent to at least 60% of the skin barrier function present in normal untreated skin based on an indicator of skin barrier function; and
applying the at least one active substance to the region of skin immediately before the treating, during the treating, and/or following the treating within the limited period of time over which the treating increases the permeability of the region of skin to the at least one active substance.

47. The method of claim 46, wherein the alterations in the epidermis and dermis further comprise a plurality of pores in the stratum corneum which extend to a depth less than the full thickness of the stratum corneum.

48. The method of claim 46, wherein the indicator of skin barrier function is measurement of transepidermal water loss.

49. The method of claim 46, wherein the indicator of skin barrier function is measurement of skin electrical resistance.

50. The method of claim 46, wherein the indicator of skin barrier function is measurement of tritiated water flux.

51. The method of claim 46, wherein the indicator of skin barrier function is measurement of skin susceptibility to an irritant.

52. The method of claim 46, wherein the indicator of skin barrier function is measurement of skin susceptibility to an infectious agent.

53. The method of claim 46, wherein an increase in a level of uptake of the active substance is dependent on the number of independent treatment zones created during the treating.

54. The method of claim 46, wherein an increase in a level of uptake of the active substance is dependent on the density of independent treatment zones created during the treating.
